(19) United States
(12) Patent Application Publication
(10) Pub. No.: US 2018/0073063 A1
(43) Pub. Date: Mar. 15, 2018

(54) REUSABLE MICROARRAY COMPOSITIONS AND METHODS

(71) Applicant: TL BIOLABS CORP., Santa Clara, CA (US)

(72) Inventor: Frederick Edward TURNER, Santa Clara, CA (US)

(73) Assignee: TL BIOLABS CORP., Santa Clara, CA (US)

(21) Appl. No.: 15/700,030

(22) Filed: Sep. 8, 2017

Related U.S. Application Data
(60) Provisional application No. 62/385,938, filed on Sep. 9, 2016.

(51) Int. Cl. C12Q 1/68

(52) U.S. Cl. CPC C12Q 1/6837; C12Q 1/6827

(57) ABSTRACT

The present application provides a reusable microarray comprising a plurality of immobilized oligonucleotide probes each having one or more nucleotides that are resistant to cleavage. The microarray is useful for analyzing nucleic acids, including single nucleotide polymorphisms (SNPs) by extension or ligation assays. The microarray can be reused by treatment that cleaves non-resistant extension or ligation products on the free termini of the oligonucleotide probes.

### CROSS-REFERENCE TO RELATED APPLICATIONS

This application claims the priority benefit of U.S. Provisional Application No. 62/385,938, filed on Sep. 9, 2016, the contents of which are hereby incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 768592000100SEQLIST.txt, date recorded: Sep. 8, 2017, size: 3 KB).

### FIELD OF THE INVENTION

The present invention relates to the field of genomics, in particular, methods and compositions for nucleic acid detection and analysis.

### BACKGROUND OF THE INVENTION

DNA microarrays are 2-dimensional arrays of DNA oligonucleotide probes attached either to a solid substrate, or to a series of beads subsequently attached to a solid substrate (bead arrays). Each oligonucleotide probe is usually between 20 and 100 nucleotides in length and can be attached to the surface either at its 3′ or 5′ end.

DNA microarrays have become a common technique used for single nucleotide polymorphism (SNP) genotyping of DNA samples. Current SNP microarrays can be classified into three major categories based on the underlying detection chemistry, including hybridization-based microarrays, extension-based microarrays (such as Illumina's INFINIUM® Assay, see, for example, Steemers F J. et. al. Nat. Methods 2006 3:31), and ligation-based microarrays (such as Affymetrix's Axiom Assay, see, for example, US20080131894). Hybridization-based microarrays detect a plurality of SNPs each designed to be in the center of a probe based on the level of hybridization of the probe to target DNA under stringent conditions. Thus, hybridization-based microarrays have the very distinct disadvantage of low accuracy for calling SNPs, particularly for highly multiplexed (>1000 SNPs) reactions. However, hybridization-based microarrays can be used more than once as the hybridized target DNA can be removed under denaturing conditions without damaging the probes (see, for example, US20070059742).

Because most SNP genotyping applications require high multiplexity and high accuracy (>95% call rate and >99.5% concordance), SNP microarrays based on enzymatic assays, such as Illumina's INFINIUM® assay and Affymetrix's AXIOM® assay, are most commonly used today. The INFINIUM® assay hybridizes the target DNA to probes each bound to a bead by its 5′ ends, and extends the free 3′ end using a polymerase when the sequence of a probe is perfectly complementary to the sequence immediately downstream of a SNP of interest. The polymerase specifically incorporates a chain-terminating modified nucleotide labelled with a hapten based on the identity of the SNP nucleotide in the target DNA. The microarray is subsequently stained with fluorescently labelled antibodies that each recognizes a specific hapten, thereby allowing detection of different SNP alleles in a highly multiplexed manner using a chip scanner. In the Affymetrix's AXIOM® assay, the target DNA is hybridized to probes attached to a solid substrate by their 3′ ends. A random adapter having a 3′ nucleotide that matches a SNP of interest and a specific 5′ hapten is then ligated to the probe-target DNA hybrid, and the hapten is detected by staining with one or more fluorescently labelled antibodies. Both enzyme-based assays are significantly more accurate at genotyping SNPs than hybridization-based assays, but both covalently modify the probes in the process of genotyping the sample. Hence, simply washing off the target DNA is not enough to reset the enzyme-based SNP microarrays and reuse is not possible. Although reagents for using microarrays are relatively cheap, the high cost associated with production of microarrays makes single-use SNP microarrays too costly for many applications such as in agriculture.

The disclosures of all publications, patents, patent applications and published patent applications referred to herein are hereby incorporated herein by reference in their entirety.

### BRIEF SUMMARY OF THE INVENTION

The present invention provides a reusable microarray and methods for use and reuse of the microarray to analyze nucleic acid samples.

One aspect of the present application provides a microarray comprising a plurality of single-stranded oligonucleotide probes each having a first terminus and a second terminus, wherein each oligonucleotide probe is attached to a solid substrate via the first terminus, wherein the terminal nucleotide at the second terminus of each oligonucleotide probe is resistant to cleavage, and wherein at least two of the oligonucleotide probes on the microarray are different. In some embodiments, the last two or more (e.g., the last 3, 4, 5, or more) nucleotides at the second terminus of each oligonucleotide probe are resistant to cleavage. In some embodiments, the terminal nucleotide at the second terminus is resistant to a nuclease. In some embodiments, the last two or more (e.g., the last 3, 4, 5, or more) nucleotides are resistant to a nuclease. In some embodiments, the nuclease is an exonuclease. In some embodiments, the first terminus is the 5′ terminus. In some embodiments, the exonuclease is a 3′ to 5′ exonuclease. In some embodiments, the first terminus is the 3′ terminus. In some embodiments, the terminal nucleotide at the second terminus is resistant to chemical cleavage. In some embodiments, the last two or more (e.g., the last 3, 4, 5, or more) nucleotides are resistant to chemical cleavage.

In some embodiments according to any one of the microarrays described above, the microarray comprises at least about 200 (such as at least about any of 400, 600, 800, 1000, 10⁴, 10⁵, 10⁶, or more) different oligonucleotide probes.

In some embodiments according to any one of the microarrays described above, the terminal nucleotide at the second terminus of each oligonucleotide probe is a modified nucleotide having a modified linkage to the penultimate nucleotide. In some embodiments, the terminal nucleotide at the second terminus is linked to the penultimate nucleotide via a phosphorothioate group. In some embodiments, the phosphorothioate group has Sp configuration. In some embodiments, the terminal nucleotide at the second terminus is linked to the penultimate nucleotide via a phosphoroselenoate group (e.g., with Sp configuration). In some embodiments, the terminal nucleotide at the second terminus is the only cleavage-resistant nucleotide in each oligonucleotide probe. In some embodiments, the last two or more (e.g., the last 3, 4, 5, or more) nucleotides at the second terminus of each oligonucleotide probe are resistant to cleavage. In some embodiments, the last two or more (e.g., the last 3, 4, 5, or more) nucleotides at the second terminus of each oligonucleotide probe are modified nucleotides having modified linkages to neighboring nucleotides. In some embodiments, the last two or more (e.g., the last 3, 4, 5, or more) nucleotides at the second terminus are each linked to their neighboring nucleotides via phosphorothioate groups (e.g., with Sp configuration). In some embodiments, the last two or more (e.g., the last 3, 4, 5, or more) nucleotides at the second terminus are each linked to their neighboring nucleotides via phosphoroselenoate groups (e.g., with Sp configuration).

In some embodiments according to any one of the microarrays described above, each oligonucleotide probe is about 20 nucleotides to about 100 nucleotides long.

In some embodiments according to any one of the microarrays described above, each oligonucleotide probe is attached to a pre-determined position on the solid substrate. In some embodiments, the plurality of oligonucleotide probes is attached to the same solid substrate. In some embodiments, each oligonucleotide probe is attached to a random position on the solid substrate. In some embodiments, the plurality of oligonucleotide probes is attached to different solid substrates.

In some embodiments according to any one of the microarrays described above, the plurality of oligonucleotide probes comprises one or more oligonucleotide probe pairs, wherein each oligonucleotide probe pair comprises a first probe and a second probe each comprising a matching (e.g., perfectly matching) sequence immediately upstream or immediately downstream of a single-nucleotide polymorphism (SNP), and wherein the terminal nucleotide at the second terminus of the first probe perfectly matches a first allele of the SNP and the terminal nucleotide at the second terminus of the second probe matches a second allele of the SNP. In some embodiments, each oligonucleotide probe pair is attached to a different solid substrate.

In some embodiments according to any one of the microarrays described above, the microarray further comprises a plurality of target nucleic acids, wherein the target nucleic acids are hybridized to at least a portion of the oligonucleotide probes.

One aspect of the present application provides a single-stranded oligonucleotide probe having a first terminus and a second terminus, wherein the oligonucleotide probe is attached to a solid substrate via the first terminus, and wherein the terminal nucleotide at the second terminus is resistant to cleavage. In some embodiments, there is provided a complex comprising the oligonucleotide probe described above and a target nucleic acid hybridized to the oligonucleotide probe.

One aspect of the present application provides a method of preparing a reusable microarray, comprising: synthesizing a plurality of single-stranded oligonucleotide probes each having a first terminus and a second terminus on a solid substrate, wherein the first terminus of each oligonucleotide probe is attached to the solid substrate, and wherein the terminal nucleotide at the second terminus of each oligonucleotide probe is resistant to cleavage, and wherein at least two of the oligonucleotide probes on the microarray are different, thereby providing the reusable microarray. In some embodiments, the terminal nucleotide at the second terminus is linked to the penultimate nucleotide by a phosphorothioate linkage or a phosphoroselenoate linkage (e.g., with Sp configuration). In some embodiments, the last two or more (e.g., the last 3, 4, 5, or more) nucleotides at the second terminus are each linked to their neighboring nucleotides via phosphorothioate groups or phosphoroselenoate groups (e.g., with Sp configuration). In some embodiments, the phosphorothioate linkage is synthesized by sulfurization of a phosphite triester linkage.

In some embodiments according to any one of the methods of preparing a reusable microarray described above, the method further comprises contacting the plurality of oligonucleotide probes with an exonuclease.

In some embodiments according to any one of the methods of preparing a reusable microarray described above, the plurality of oligonucleotide probes comprises at least about 200 (such as at least about any one of 400, 600, 800, 1000, 2000 or more) different oligonucleotide probes.

One aspect of the present application provides an extension-based method of detecting the presence or absence of a single-nucleotide polymorphism (SNP) allele in a target nucleic acid, comprising: (a) hybridizing the target nucleic acid to the oligonucleotide probes of any one of the microarrays described above to provide probe-target hybrids, wherein the first terminus is the 5′ terminus, and wherein at least one oligonucleotide probe comprises a sequence that matches (e.g., perfectly matches) the SNP allele; (b) contacting the probe-target hybrids with a polymerase and nucleotides under a condition that allows primer extension to provide modified probe-target hybrids; and (c) detecting the modified probe-target hybrids thereby detecting the presence or absence of the SNP allele in the target nucleic acid. In some embodiments, the polymerase is a polymerase with proofreading activity.

In some embodiments according to any one of the extension-based methods described above, the primer extension is allele-specific primer extension (ASPE). In some embodiments, the nucleotides are fluorescently labelled, and wherein the modified probe-target hybrids are detected by detecting fluorescence from the nucleotides incorporated in the modified probe-target hybrids. In some embodiments, step (c) comprises contacting the modified probe-target hybrids with a dye that specifically binds to double-stranded nucleic acids and detecting signal from the dye that is bound to the modified probe-target hybrids. In some embodiments, step (c) comprises subjecting the modified probe-target hybrids to a denaturing condition to provide modified probes, contacting the modified probes with a dye that specifically binds to single-stranded nucleic acids, and detecting signal from the dye that is bound to the modified probes. In some embodiments, the denaturing condition is chemical denaturation and/or heat denaturation.

In some embodiments according to any one of the extension-based methods described above, the primer extension is single base extension (SBE). In some embodiments, the nucleotides are chain-terminating nucleotides comprising a hapten. In some embodiments, step (c) comprises contacting the modified probe-target hybrids with a fluorescently labelled protein that specifically binds to the hapten, and detecting the fluorescently labelled protein attached to the modified probe-target hybrids.

One aspect of the present application provides a ligation-based method of detecting the presence or absence of a single-nucleotide polymorphism (SNP) allele in a target nucleic acid, comprising: (a) hybridizing the target nucleic acid to the oligonucleotide probes of any one of the microarrays described above to provide probe-target hybrids, wherein at least one oligonucleotide probe comprises a sequence that matches (e.g., perfectly matches) the SNP allele; (b) contacting the probe-target hybrids with a ligase in the presence of a plurality of free adapter oligonucleotides under a condition that allows allele-specific ligation to provide modified probe-target hybrids; and (c) detecting the modified probe-target hybrids, thereby detecting the presence or absence of the SNP allele in the target nucleic acid. In some embodiments, each free adapter oligonucleotide is labelled with a fluorophore or a hapten.

In some embodiments according to any one of the extension-based or ligation-based methods described above, the method further comprises (d) treating the microarray by contacting the microarray with an exonuclease. In some embodiments, the treating further comprises subjecting the microarray to a denaturing condition. In some embodiments, the denaturing condition is chemical denaturation and/or heat denaturation. In some embodiments, the method further comprises (e) reusing the microarray according to steps (a)-(c). In some embodiments, the microarray is reused for about 2 times to about 100 times.

It is understood that aspects and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

These and other aspects and advantages of the present invention will become apparent from the subsequent detailed description and the appended claims. It is to be understood that one, some, or all of the properties of the various embodiments described herein may be combined to form other embodiments of the present invention just as if each and every combination is individually and explicitly disclosed.

### BRIEF DESCRIPTION OF THE DRAWINGS

FIG. 1 illustrates an exemplary oligonucleotide probe pair used in a method to detect an SNP allele using a 1-color allele-specific primer extension (ASPE) assay. Fluorescently labelled nucleotides are used in this method for detection of the extended oligonucleotide probe.

FIG. 2 illustrates an exemplary oligonucleotide probe pair used in a method to detect an SNP allele using a 1-color single base extension (SBE) assay. Hapten-labelled chain-terminating nucleotides are used in this method for detection of the extended oligonucleotide probe.

FIG. 3 illustrates an exemplary oligonucleotide probe used in a method to detect a SNP using a 2-color single base extension (SBE) assay. Hapten-labelled chain-terminating nucleotides are used in this method for detection of the extended oligonucleotide probe. ddATP and ddGTP are labelled with a first hapten, and ddCTP and ddTTP are labelled with a second hapten.

FIG. 4 illustrates an exemplary oligonucleotide probe pair used in a method to detect an SNP allele using a 1-color allele-specific ligation assay, in which the oligonucleotide probes are attached to the solid substrate via their 3′ termini. A pool of hapten or fluorescently labelled free adapter oligonucleotides having random sequences is used in this method for detection of the ligated oligonucleotide probe.

FIG. 5 illustrates an exemplary oligonucleotide probe pair used in a method to detect an SNP allele using a 1-color allele-specific ligation assay, in which the oligonucleotide probes are attached to the solid substrate via their 5′ termini. A pool of hapten or fluorescently labelled free adapter oligonucleotides having random sequences is used in this method for detection of the ligated oligonucleotide probe.

FIG. 6 illustrates an exemplary oligonucleotide probe used in a method to detect an SNP allele using a 2-color allele specific ligation assay, in which the oligonucleotide probe is attached to the solid substrate via their 3′ termini. Two pools of hapten or fluorescently labelled free adapter oligonucleotides each having the SNP nucleotide followed by a random sequence are used in this method for detection of the ligated oligonucleotide probe. Each pool has a different label or a different fluorophore.

FIG. 7 illustrates an exemplary oligonucleotide probe used in a method to detect an SNP allele using a 2-color allele specific ligation assay, in which the oligonucleotide probe is attached to the solid substrate via their 5′ termini. Two pools of hapten or fluorescently labelled free adapter oligonucleotides each having the SNP nucleotide followed by a random sequence are used in this method for detection of the ligated oligonucleotide probe. Each pool has a different label or a different fluorophore.

FIG. 8 illustrates an exemplary oligonucleotide probe pair used in a method to detect an SNP allele using a 1-color allele-specific primer extension (ASPE) assay. Specific DNA dyes (ssDNA-binding dye or dsDNA binding dye) are used for detection of extended oligonucleotide probes.

FIG. 9 illustrates use and reuse of the microarray described in the present application. This reuse process is applicable to all methods of detecting SNPs shown in FIGS. 1-8.

FIG. 10 illustrates the layout of probes on the glass slide of Example 1.

FIG. 11 shows images of probes hybridized to template DNA after extension and stripping steps (left), and after exonuclease-treatment step (right).

FIG. 12 shows quantification of fluorescence intensity of each probe spot in FIG. 11.

FIG. 13A illustrates the workflow of the experimental procedure in Example 2.

FIG. 13B illustrates the layout of probes on the glass slide used in section A of Example 2.

FIG. 14 shows images of slides after various extension and exonuclease treatment steps in section A of Example 2.

FIG. 15 illustrates the layout of probes on the glass slide used in section B of Example 2.

FIG. 16A-16E show images of slides after various extension and exonuclease treatment steps in section B of Example 2.

FIG. 17A-17E show images of slides after various extension and exonuclease treatment steps in section C of Example 2.

FIG. 18 illustrates the layout of probes on the glass slide used in Example 3.

FIG. 19 show images of slides after various extension and exonuclease treatment steps in Example 3.

### DETAILED DESCRIPTION OF THE INVENTION

The present application provides a reusable microarray comprising a plurality of immobilized oligonucleotide probes each having one or more cleavage-resistant terminal nucleotides at the free terminal end. While useful in a variety of applications, the microarray is particularly suitable for detecting single nucleotide polymorphisms (SNPs) using enzymatic assays, such as primer extension and ligation, in a highly multiplexed manner. In some embodiments, only the terminal nucleotide at the free terminus of each oligonucleotide probe is resistant to cleavage, and the terminal nucleotide at the free terminus matches the SNP nucleotide in a SNP allele of interest. In some embodiments, the last two or more (e.g., the last 3, 4, 5, or more) nucleotides at the free terminus of each oligonucleotide probe are resistant to cleavage. As the microarray is pretreated or regenerated by cleaving (e.g., by chemical or exonuclease treatment) from the free terminus, any non-resistant nucleotides, such as damaged oligonucleotide probes or extension or ligation products, only oligonucleotide probes having an intact terminal oligonucleotide are present in the microarray during a subsequent SNP detection experiment, thereby reducing noise from non-SNP specific extension or ligation events. The microarray compositions and methods of use and reuse described herein substantially reduce the cost of SNP genotyping, because the high cost associated with production of the microarray itself can be spread over many samples, while the reagent cost per sample is very low. For example, reagents for a primer extension assay may include only two enzymes (i.e., polymerase and exonuclease), and fluorescently labelled nucleotides.

Accordingly, one aspect of the present application provides a microarray comprising a plurality of single-stranded oligonucleotide probes each having a first terminus and a second terminus, wherein each oligonucleotide probe is attached to a solid substrate via the first terminus (such as the 5′ terminus), wherein the terminal nucleotide at the second terminus (such as the 3′ terminus) of each oligonucleotide probe is resistant to cleavage, and wherein at least two of the oligonucleotide probes on the microarray are different. In some embodiments, the last two or more (e.g., the last 3, 4, 5, or more) nucleotides at the second terminus of each oligonucleotide probe are resistant to cleavage.

In some embodiments, there is provided a SNP microarray comprising a plurality of single-stranded oligonucleotide probe pairs each comprising a first probe and a second probe, wherein each probe has a first terminus and a second terminus, wherein each probe is attached to a solid substrate via the first terminus (such as the 5′ terminus), wherein the terminal nucleotide at the second terminus (such as the 3′ terminus) of each probe is resistant to cleavage, wherein the first probe and the second probe each comprises a matching (e.g., perfectly matching) sequence immediately upstream or immediately downstream of a single-nucleotide polymorphism (SNP), and wherein the terminal nucleotide at the second terminus of the first probe matches a first allele of the SNP and the terminal nucleotide at the second terminus of the second probe matches a second allele of the SNP. In some embodiments, the last two or more (e.g., the last 3, 4, 5, or more) nucleotides at the second terminus of each oligonucleotide probe are resistant to cleavage.

One aspect of the present application comprises a method of detecting the presence or absence of a single-nucleotide polymorphism (SNP) allele in a sample of target nucleic acids, comprising: (a) hybridizing the target nucleic acid to the oligonucleotide probes of the SNP microarray described herein to provide probe-target hybrids, wherein the first terminus is the 5′ terminus, and wherein at least one oligonucleotide probe pair comprises a sequence that matches (e.g., perfectly matches) the SNP allele; (b) contacting the probe-target hybrids with a polymerase and nucleotides under a condition that allows primer extension to provide modified probe-target hybrids; and (c) detecting the modified probe-target hybrids, thereby detecting the presence or absence of the SNP allele in the target nucleic acid. In some embodiments, the last two or more (e.g., the last 3, 4, 5, or more) nucleotides at the second terminus of each oligonucleotide probe are resistant to cleavage.

### Microarray

One aspect of the present application provides a microarray comprising a plurality of single-stranded oligonucleotide probes each having a first terminus and a second terminus, wherein each oligonucleotide probe is attached to a solid substrate via the first terminus, wherein the terminal nucleotide at the second terminus of each oligonucleotide probe is resistant to cleavage, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the terminal nucleotide at the second terminus is the only cleavage-resistant nucleotide in each oligonucleotide probe. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes each having a first terminus and a second terminus, wherein each oligonucleotide probe is attached to a solid substrate via the first terminus, wherein the last two or more (e.g., the last 3, 4, 5 or more) nucleotides at the second terminus of each oligonucleotide probe are resistant to cleavage, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the last two or more (e.g., the last 3, 4, 5 or more) nucleotides at the second terminus are the only cleavage-resistant nucleotides in each oligonucleotide probe. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

Each oligonucleotide probe may be attached to the solid substrate via the 5′ terminus or the 3′ terminus. Thus, in some embodiments, there is provided a microarray comprising a plurality of single-stranded oligonucleotide probes, wherein each oligonucleotide probe is attached to a solid substrate via the 5′ terminus, wherein the 3′ terminal nucleotide of each oligonucleotide probe is resistant to cleavage, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 3′ terminal nucleotide is the only nucleotide in each oligonucleotide probe that is resistant to resistant to cleavage, e.g., chemical cleavage or cleavage by nuclease. In some embodiments, the last two or more (e.g., the last 3, 4, 5 or more) nucleotides at the 3′ terminus of each oligonucleotide probe are resistant to cleavage, e.g., chemical cleavage or cleavage by nuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide of each oligonucleotide probe is resistant to cleavage, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 5′ terminal nucleotide is the only nucleotide in each oligonucleotide probe that is resistant to cleavage, e.g., chemical cleavage or cleavage by nuclease. In some embodiments, the last two or more (e.g., the last 3, 4, 5 or more) nucleotides at the 5′ terminus of each oligonucleotide probe are resistant to cleavage, e.g., chemical cleavage or cleavage by nuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 5′ terminus, wherein the 3′ terminal nucleotide of each oligonucleotide probe is resistant to a nuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 3′ terminal nucleotide is the only nucleotide in each oligonucleotide probe that is resistant to the nuclease. In some embodiments, the last two or more (e.g., the last 3, 4, 5 or more) nucleotides at the 3′ terminus of each oligonucleotide probe are resistant to the nuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide of each oligonucleotide probe is resistant to a nuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 5′ terminal nucleotide is the only nucleotide in each oligonucleotide probe that is resistant to the nuclease. In some embodiments, the last two or more (e.g., the last 3, 4, 5 or more) nucleotides at the 5′ terminus of each oligonucleotide probe are resistant to the nuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 5′ terminus, wherein the 3′ terminal nucleotide of each oligonucleotide probe is resistant to a 3′ to 5′ exonuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 3′ terminal nucleotide is the only nucleotide in each oligonucleotide probe that is resistant to the 3′ to 5′ exonuclease. In some embodiments, the last two or more (e.g., the last 3, 4, 5 or more) nucleotides at the 3′ terminus of each oligonucleotide probe are resistant to the 3′ to 5′ exonuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide of each oligonucleotide probe is resistant to a 5′ to 3′ exonuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, the 5′ terminal nucleotide is the only nucleotide in each oligonucleotide probe that is resistant to the 5′ to 3′ exonuclease. In some embodiments, the last two or more (e.g., the last 3, 4, 5 or more) nucleotides at the 5′ terminus of each oligonucleotide probe are resistant to the 5′ to 3′ exonuclease. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a modified linkage (e.g., phosphorothioate or phosphoroselenoate) in each oligonucleotide probe, and wherein at least two of the oligonucleotide probes on the microarray are different. In some embodiments, the last two or more (e.g., the last 3, 4, 5 or more) nucleotides at the 3′ terminus of each oligonucleotide probe are each linked to their neighboring nucleotides via modified linkages (e.g., phosphorothioate or phosphoroselenoate). In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a modified linkage (e.g., phosphorothioate or phosphoroselenoate) in each oligonucleotide probe, and wherein at least two of the oligonucleotide probes on the microarray are different. In some embodiments, the last two or more (e.g., the last 3, 4, 5 or more) nucleotides at the 5′ terminus of each oligonucleotide probe are each linked to their neighboring nucleotides via modified linkages (e.g., phosphorothioate or phosphoroselenoate). In some embodiments, at least about 200 (such as at least about any of 400, 600, 800, 1000, 10⁴, 10⁵, 10⁶ or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group (such as the Sp stereoisomer) in each oligonucleotide probe, and wherein at least two of the oligonucleotide probes on the microarray are different. In some embodiments, the last two or more (e.g., the last 3, 4, 5 or more) nucleotides at the 3′ terminus of each oligonucleotide probe are each linked to their neighboring nucleotides via phosphorothioate groups (such as the Sp stereoisomer). In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a phosphorothioate group (such as the Sp stereoisomer) in each oligonucleotide probe, and wherein at least two of the oligonucleotide probes on the microarray are different. In some embodiments, the last two or more (e.g., the last 3, 4, 5 or more) nucleotides at the 5′ terminus of each oligonucleotide probe are each linked to their neighboring nucleotides via phosphorothioate groups (such as the Sp stereoisomer). In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group in the Sp configuration in each oligonucleotide probe, wherein the 3′ terminal nucleotide is the only nucleotide in each oligonucleotide probe that is resistant to a 3′ to 5′ exonuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

In some embodiments, there is provided a microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a phosphorothioate group in the Sp configuration in each oligonucleotide probe, wherein the 5′ terminal nucleotide is the only nucleotide in each oligonucleotide probe that is resistant to a nuclease, and wherein at least two of the oligonucleotide probes on the microarray are different. In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

Further provided is a used microarray comprising any one of the microarrays described above, further comprising a plurality of target nucleic acids, wherein the target nucleic acids are hybridized to at least a portion of the oligonucleotide probes. In some embodiments, at least a portion of the oligonucleotide probes are further extended at the free terminus by one or more nucleotides, or covalently ligated to nucleic acids at the free terminus. The used microarray may be regenerated by treatment with the cleavage method (such as exonuclease treatment), and/or by denaturation.

The microarrays provided herein include arrays or patterns of oligonucleotide probes in any suitable format. In some embodiments, the oligonucleotide probes are deposited as small spots in a test area on a single solid substrate. “Test area” refers to any surface area corresponding to and immediately surrounding an array within the microarray. For example, “test area” includes surface area within a well in a microplate or the surface area of a glass microscopic slide or the surface area of a bead wherein the spots are deposited as an array. The test areas may be separated physically, for example, by wells, raised regions, pins, etched trenches, or the like. In some embodiments, different oligonucleotide probes are deposited in different test areas on the microarray. In some embodiments, each test area comprises a plurality of the oligonucleotide probes having the same sequences. Each test area may comprise at least about any of 1, 2, 5, 10, 20, 50, 100, 200, 500, 1000 or more molecules of the oligonucleotide probes.

In some embodiments, the plurality of oligonucleotide probes is attached to the same solid substrate. For example, the plurality of oligonucleotide probes is attached to a glass slide. In some embodiments, each oligonucleotide probe is attached at a pre-determined position (i.e., pre-determined test area) on the solid substrate. The positional information of each oligonucleotide probe on the microarray allows the user to retrieve the sequence information of each oligonucleotide probe. In some embodiments, the microarray comprises more than one (such as about any of 2, 3, 4, 5, 6, 7, 8, 9, 10, 15, 20, 25, or more) distinct test areas that comprise the same oligonucleotide probe. Test areas with the same oligonucleotide probe on the microarray provide experimental replicate data, which may be averaged or analyzed statistically to enhance signal to noise ratio and improve accuracy. In some embodiments, the microarray comprises reference oligonucleotide probes in certain test areas, which can serve as experimental controls, and to allow calibration of the background signal.

In some embodiments, the plurality of oligonucleotide probes is attached to different solid substrates. For example, each oligonucleotide probe or each group of oligonucleotide probes (e.g., SNP oligonucleotide probe pair) is attached to a bead. In some embodiments, the solid substrate is further disposed in a compartment of a solid carrier, such as a multi-well plate, or a fiber optic bundle, or a glass slide. In some embodiments, each oligonucleotide probe is attached to a random position on the solid substrate. In some embodiments, the position of each oligonucleotide probe or the solid substrate associated thereof on the solid carrier may be determined prior to using the microarray, thereby allowing retrieval of the sequence information of the oligonucleotide probe based on the position of the oligonucleotide probe on the solid carrier.

The microarray may comprise any number of different oligonucleotide probes. As used herein, different oligonucleotide probes comprise different sequences, and thus can be used to detect different loci or alleles. In some embodiments, the microarray comprises at least about any of 2, 5, 10, 20, 50, 100, 200, 400, 500, 600, 800, 1000, 2000, 5000, 10⁴, 2×10⁴, 5×10⁴, 10⁵, 2×10⁵, 5×10⁵, 10⁶, 2×10⁶, 5×10⁶, or 10⁷ different oligonucleotide probes. In some embodiments, the microarray comprises any one of about 2-10, 10-100, 200-400, 100-1000, 1000-10⁴, 10⁴-10⁵, 10⁵-10⁶, 10⁶-10⁷, 2-10³, 10³-10⁵, 10⁵-10⁷, or 2-10⁷ different oligonucleotide probes. In some embodiments, the microarray is a high-density array.

### Oligonucleotide Probe

The microarrays of the present application are arrays or patterns of immobilized oligonucleotide probes.

In some embodiments, the present application provides a single-stranded oligonucleotide probe (such as DNA probe) having a first terminus and a second terminus, wherein the oligonucleotide probe is attached to a solid substrate via the first terminus, and wherein the terminal nucleotide at the second terminus is resistant to cleavage. In some embodiments, the terminal nucleotide at the second terminus is the only nucleotide in the oligonucleotide probe that is resistant to cleavage. In some embodiments, the last two or more (e.g., the last 3, 4, 5 or more) nucleotides are resistant to cleavage. In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 5′ terminus, and wherein the 3′ terminal nucleotide is resistant to cleavage. In some embodiments, the 3′ terminal nucleotide is resistant to chemical cleavage. In some embodiments, the 3′ terminal nucleotide is resistant to cleavage by a nuclease, such as an exonuclease, e.g., a 3′ to 5′ exonuclease. In some embodiments, the 3′ terminal nucleotide is the only nucleotide in the oligonucleotide probe that is resistant to cleavage by the nuclease. In some embodiments, the last two or more (e.g., the last 3, 4, 5 or more) nucleotides at the 3′ terminus are resistant to cleavage by the nuclease, such as a 3′ to 5′ exonuclease. In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 3′ terminus, and wherein the 5′ terminal nucleotide is resistant to cleavage. In some embodiments, the 5′ terminal nucleotide is resistant to chemical cleavage. In some embodiments, the last two or more (e.g., the last 3, 4, 5 or more) nucleotides at the 5′ terminus are resistant to chemical cleavage. In some embodiments, the 5′ terminal nucleotide is resistant to cleavage by a nuclease, such as an exonuclease, for example, a 5′ to 3′ exonuclease. In some embodiments, the 5′ terminal nucleotide is the only nucleotide in the oligonucleotide probe that is resistant to cleavage by the nuclease. In some embodiments, the last two or more (e.g., the last 3, 4, 5 or more) nucleotides at the 5′ terminus are resistant to cleavage by the nuclease, such as 5′ to 3′ exonuclease. In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, a single-stranded oligonucleotide probe (such as DNA probe), wherein the oligonucleotide probe is attached to a solid substrate via the 5′ terminus, wherein the 3′ terminal nucleotide is linked to the penultimate nucleotide via a modified linkage (e.g., phosphorothioate or phosphoroselenoate). In some embodiments, the last two or more (e.g., the last 3, 4, 5 or more) nucleotides at the 3′ terminus are each linked to their neighboring nucleotides via modified linkages (e.g., phosphorothioate or phosphoroselenoate). In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, a single-stranded oligonucleotide probe (such as DNA probe), wherein the oligonucleotide probe is attached to a solid substrate via the 3′ terminus, wherein the 5′ terminal nucleotide is linked to the penultimate nucleotide via a modified linkage (e.g., phosphorothioate or phosphoroselenoate). In some embodiments, the last two or more (e.g., the last 3, 4, 5 or more) nucleotides at the 5′ terminus are each linked to their neighboring nucleotides via modified linkages (e.g., phosphorothioate or phosphoroselenoate). In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 5′ terminus, and wherein the 3′ terminal nucleotide is linked to the penultimate nucleotide via a phosphorothioate group (such as the Sp stereoisomer). In some embodiments, the 3′ terminal nucleotide is resistant to cleavage by an exonuclease, for example, a 3′ to 5′ exonuclease. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 3′ terminus, and wherein the 5′ terminal nucleotide is linked to the penultimate nucleotide via a phosphorothioate group (such as the Sp stereoisomer). In some embodiments, the 5′ terminal nucleotide is resistant to cleavage by a nuclease, such as an exonuclease, for example, a 5′ to 3′ exonuclease. In some embodiments, the last two or more (e.g., the last 3, 4, 5 or more) nucleotides at the 5′ terminus are each linked to their neighboring nucleotides via phosphorothioate groups (such as the Sp stereoisomer). In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 5′ terminus, wherein the 3′ terminal nucleotide is linked to the penultimate nucleotide via a phosphorothioate group in the Sp configuration, and wherein the 3′ terminal nucleotide is the only nucleotide in the oligonucleotide probe that is resistant to a 3′ to 5′ exonuclease. In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

In some embodiments, there is provided a single-stranded oligonucleotide probe (such as DNA probe), wherein the oligonucleotide probe is attached to a solid substrate via the 3′ terminus, wherein the 5′ terminal nucleotide is linked to the penultimate nucleotide via a phosphorothioate group in the Sp configuration, and wherein the 5′ terminal nucleotide is the only nucleotide in the oligonucleotide probe that is resistant to a 5′ to 3′ exonuclease. In some embodiments, the oligonucleotide probe is about 20 to about 100 nucleotides long. In some embodiments, the solid substrate is a glass slide. In some embodiments, the oligonucleotide probe is attached to a pre-determined position on the solid substrate. In some embodiments, the solid substrate is a bead. In some embodiments, the oligonucleotide probe is attached to a random position on the solid substrate.

Also provided is a complex comprising any one of the oligonucleotide probes described herein comprising a target nucleic acid hybridized to the oligonucleotide probe. In some embodiments, the oligonucleotide probe is extended by one or more nucleotides at the terminus not attached to the solid substrate. In some embodiments, the oligonucleotide probe is ligated to a nucleic acid. The oligonucleotide probe in the complex may be regenerated by treatment with the cleavage method, such as exonuclease treatment, and/or by denaturation.

The oligonucleotide probes, including individual oligonucleotide probes and oligonucleotide probes on any of the microarrays described herein, are single-stranded nucleic acids having a 5′ terminus and a 3′ terminus, in which a first terminus is attached to a solid substrate, and the second terminus is free, and thus can be modified or manipulated in an assay subsequent to hybridization of a target nucleic acid to the oligonucleotide probe. The second terminus is also referred herein as the “free terminus.”

The oligonucleotide probe may have any suitable length based on factors, including, but not limited to desired binding specificity, melting temperature, secondary structures, and complexity of the target nucleic acid. For example, for a target nucleic acid with relatively high complexity, i.e., a relatively large total length of unique sequence, the oligonucleotide probe is designed to contain a relatively longer sequence to avoid nonspecific binding. The oligonucleotide probe is also designed to have a suitable length to allow hybridization to the target nucleic acid under suitable experimental conditions (i.e., in a suitable temperature range, at suitable ionic strength, and within a suitable time such as about 30 minutes to about 48 hours). Longer oligonucleotide probes may be chosen to enhance the specificity of hybridization, but too long of a sequence may lead to undesirable consequences, such as binding to partial complements, formation of internal secondary structures, or difficulty in dissociating the target nucleic acid from the oligonucleotide probe. In some embodiments, the oligonucleotide probe is at least about any one of 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100 nucleotides long. In some embodiments, the oligonucleotide probe is about any one of 20-30, 30-40, 25-50, 50-75, 75-100, 20-40, 20-50, 50-100, 20-60, 20-80, or 20-100 nucleotides long. In some embodiments, the oligonucleotide probe is about 24 nucleotides long.

The sequence of the oligonucleotide probe is designed to match (e.g., perfectly match) an allele of interest, or a diagnostic portion thereof. As used herein, a “matching” sequence refers to a sequence that is substantially identical to the coding strand sequence of the allele or diagnostic portion thereof, or a sequence that is substantially complementary to the coding strand sequence (i.e., identical to the noncoding strand sequence) of the allele or diagnostic portion thereof, and wherein the sequence is specific to the allele or a diagnostic portion thereof, as compared to a different allele or diagnostic portion thereof. A “perfectly matching” sequence refers to a sequence that is identical to the coding strand sequence of the allele or diagnostic portion thereof, or a sequence that is complementary to the coding strand sequence (i.e., identical to the noncoding strand sequence) of the allele or diagnostic portion thereof. Thus, an oligonucleotide probe comprising a matching (e.g., perfectly matching) sequence for an allele of interest can hybridize to either the coding strand or the noncoding strand of the allele. A matching sequence may contain one or more mismatches at positions that do not differ among the different alleles, and the mismatches do not affect specific hybridization of the matching sequence to the allele. For example, a matching sequence of an SNP allele may contain one or more mismatches at positions other than the SNP nucleotide position. A “substantially” identical sequence is a sequence that is at least about 70%, 75%, 80%, 85%, 90%, 95%, 98%, 99%, or more identical to the sequence in comparison. A “substantially” complementary sequence is a sequence that is at least about 70%, 75%, 80%, 85%, 90%, 95%, 98%, 99%, or more complementary to the sequence in comparison. In some embodiments, a matching sequence of an SNP allele has no more than about any one of 5, 4, 3, 2, or 1 mismatch at position(s) other than the SNP nucleotide. In some embodiments, a matching sequence of an SNP allele has only 1 or 2 mismatches at position(s) other than the SNP nucleotide. Unless stated otherwise, sequences described herein are compared to the coding strand sequence of an allele of interest or diagnostic portion thereof. An oligonucleotide probe comprising the substantially identical (e.g., completely identical) sequence as the allele hybridizes to the noncoding strand of the allele, and an oligonucleotide probe comprising the substantially complementary (e.g., completely complementary) sequence of the allele hybridizes to the coding strand of the allele.

Exemplary diagnostic portions include, for example, nucleic acid sequences adjacent to or near, including, for example, immediately upstream (i.e., 5′ to) or immediately downstream (i.e., 3′ to) of, a typable locus in the allele. “Typable locus” refers to a location of genetic variation in an allele of interest, including, for example, single nucleotide polymorphisms (SNPs), mutations, variable number of tandem repeats (VNTRs) and single tandem repeats (STRs), other polymorphisms, insertions, deletions, splice variants or any other known genetic markers. Design of the sequence of oligonucleotide probes is accomplished using practices which are standard in the art. For example, sequences that have self-complementarity, such that the resulting oligonucleotides would either fold upon themselves, or hybridize to each other at the expense of binding to the target nucleic acid, are generally avoided.

The sequence of the oligonucleotide probe can be designed based on the known sequence information of SNPs and other genetic variations in public databases. In some embodiments, the oligonucleotide probe comprises a typable locus at the free terminus. For example, in some embodiments, the terminal nucleotide at the free terminus of the oligonucleotide probe corresponds to a SNP. In some embodiments, the oligonucleotide probe comprises a matching (e.g., perfectly matching) sequence immediately upstream of a typable locus (such as SNP). In some embodiments, the oligonucleotide probe comprises a matching (e.g., perfectly matching) sequence immediately downstream of a typable locus (such as SNP). The immediately upstream sequence or the immediately downstream sequence is chosen over the other to provide an oligonucleotide probe to enhance its hybridization properties, such as desirable specificity, suitable G/C content, and/or to reduce internal secondary structure. The directionality of the oligonucleotide probe sequence (i.e., whether to be the same or complementary sequence as the coding sequence of the allele) may depend on whether the upstream or downstream sequence is chosen, and on which terminus (5′ or 3′) the oligonucleotide probe is attached to the solid substrate.

The oligonucleotide probe may comprise deoxyribonucleotides (DNA), ribonucleotides (RNA), or modified nucleotides thereof (e.g., nucleic acids containing modified bases, modified phosphate linkage, modified sugar moieties, labels, binding moieties, spacers, linkers, etc.). In some preferred embodiments, the oligonucleotide probe is a DNA probe.

In some embodiments, the oligonucleotide probe comprises one or more nucleotides containing a non-natural sugar moiety in the backbone. Exemplary sugar modifications include but are not limited to 2′ modifications such as addition of halogen, alkyl, substituted alkyl, allcaryl, arallcyl, O-allcaryl or O-aralkyl, SH, SCH₃, OCN, Cl, Br, CN, CF₃, OCF₃, SOCH₃, SO₂, CH₃, ONO₂, NO₂, N₃, NH₂, heterocycloallcyl, heterocycloallcaryl, aminoallcylamino, polyallcylamino, substituted silyl, and the like. Similar modifications can also be made at other positions on the sugar, such as the 3′ position of the sugar moiety. In some embodiments, the oligonucleotide probe comprises one or more morpholino nucleotides. In some embodiments, the oligonucleotide probe comprises one or more peptide nucleic acid (PNA) nucleotides.

The oligonucleotide probe may comprise native or non-native bases. For example, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in the oligonucleotide probe, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702. In some embodiments, the oligonucleotide probe comprises one or more Locked Nucleic Acid (LNA) nucleotides or similar structures. In some embodiments, LNA nucleotides at the free terminus of the oligonucleotide probes can promote exonuclease resistance, and SNP calling accuracy.

Importantly, the terminal nucleotide at the second terminus (also referred herein as the “free terminus”) of the oligonucleotide probe is resistant to cleavage by a suitable cleavage method. In some embodiments, the entire oligonucleotide probe is resistant to the cleavage method. In some embodiments, the last two or more nucleotides, such as any one of the last 2, 3, 4, 5, 6, 7, 8, 10, 12, or more nucleotides at the second terminus of the oligonucleotide probe are resistant to cleavage by a suitable cleavage method. In some embodiments, the only nucleotide that is resistant to the cleavage method is the terminal nucleotide at the free terminus. In some embodiments, the only nucleotides that are resistant to the cleavage method are the last two or more (e.g., last four or more) nucleotides at the free terminus. For example, in some embodiments, the oligonucleotide probe is a naturally occurring nucleic acid (such as DNA) having one or more modified nucleotides at the free terminus, wherein the modified nucleotides are resistant to the cleavage method, while the rest of the oligonucleotide probe is readily cleaved by the method. In some embodiments, the terminal nucleotide (or the last two or more nucleotides) at the second terminus is resistant to cleavage by a chemical agent, which readily cleaves naturally occurring nucleic acids (such as DNA or RNA). In some embodiments, the terminal nucleotide (or the last two or more nucleotides) at the second terminus is resistant to cleavage by a nuclease, which readily cleaves naturally occurring nucleic acids (such as DNA or RNA). In some embodiments, the nuclease is an exonuclease. In some embodiments, the exonuclease cleaves double-stranded or single-stranded nucleic acids (such as dsDNA and ssDNA). In some embodiments, the exonuclease specifically cleaves single-stranded DNA (ssDNA). In some embodiments, the exonuclease is a 5′ to 3′ exonuclease. Exemplary 5′ to 3′ exonuclease include, but are not limited to, lambda Exonuclease, Exonuclease VII, RecBCD (Exonuclease V), Rec J exonuclease, and T5 exonuclease. In some embodiments, the exonuclease is a 3′ to 5′ exonuclease. Exemplary 3′ to 5′ exonuclease include, but are not limited to, Exonuclease I, Exonuclease III, Exonuclease T, RecBCD (Exonuclease V), Exonuclease VI, Exonuclease VII, and BAL-31. In some embodiments, the exonuclease can cleave naturally occurring nucleic acids or modified nucleotides that are not resistant to cleavage (e.g., those containing a fluorescent label or a hapten) in either 5′ to 3′ or 3′ to 5′ direction.

In some embodiments, the terminal nucleotide (or the last two or more nucleotides) at the second terminus is a modified nucleotide having a modified sugar moiety. In some embodiments, the terminal nucleotide (or the last two or more nucleotides) at the second terminus is a modified nucleotide having a modified linkage to the neighboring nucleotide. In some embodiments, the terminal nucleotide (or the last two or more nucleotides) at the second terminus does not inhibit polymerase activity. In some embodiments, the terminal nucleotide (or the last two or more nucleotides) at the second terminus does not inhibit ligase activity. For example, the terminal nucleotide (or the last two or more nucleotides) at the second terminus may have a substitution group at the 2′ position of the sugar moiety, such as O-methyl, or amino group, or have a modification at one or more bridging or non-bridging (positions in the phosphate group linking the terminal nucleotide (or the last two or more nucleotides) at the second terminus and its neighboring nucleotide, such as phosphorothioate group. The substitution group or the modification at the phosphate group may sterically hinder and inhibit the cleavage activity of the exonuclease and/or the exonuclease activity of a polymerase, but at the same time, allow primer extension by the polymerase or allow ligation by a ligase. Exemplary backbone modifications suitable for use in the oligonucleotide probes include, but are not limited to, N3′ phosphoramidite (NP), phosphodithioate, boronophosphate, 2′-5′-phosphodiester, amide, phosphonoacetate (PACE), phosphothioacetate (thio-PACE), Peptide Nucleic Acid (PNA) and Locked Nucleic Acid (LNA).

In some embodiments, the terminal nucleotide (or the last two or more nucleotides) at the second terminus has a stereochemically pure modified linkage. For example, the Rp stereoisomer of phosphorothioate is sensitive to exonuclease cleavage, but the Sp stereoisomer of phosphorothioate is resistant to exonuclease cleavage. In some embodiments, the terminal nucleotide at the second terminus is linked to the penultimate nucleotide via the Sp stereoisomer of phosphorothioate. In some embodiments, the last two or more (e.g., the last 3, 4, 5 or more) nucleotides at the second terminus are each linked to their neighboring nucleotides via the Sp stereoisomer of phosphorothioate.

In some embodiments, the terminal nucleotide (or the last two or more nucleotides) has a phosphoroselenoate linkage. In some embodiments, a phosphoroselenoate linkage can be used as a direct replacement for a phosphorothioate linkage in the oligonucleotide probes, as phosphoroselenoate groups in general have very similar chemical properties as phosphorothioates. See, for example, Tram K. et al. Org. Lett. 9(24): 5103-5106 (2007). In some embodiments, the phosphoroselenoate group is more resistant to exonuclease than phosphorothioate groups. In some embodiments, the terminal nucleotide at the second terminus is linked to the penultimate nucleotide via the Sp stereoisomer of phosphoroselenoate. In some embodiments, the last two or more (e.g., the last 3, 4, 5 or more) nucleotides at the second terminus are each linked to their neighboring nucleotides via the Sp stereoisomer of phosphoroselenoate.

The oligonucleotide probe is attached to the solid substrate via either its 5′ terminus or its 3′ terminus. All oligonucleotide probes on a microarray are attached to the solid substrate(s) via the same termini (i.e., all 5′ termini, or all 3′ termini). The choice of attachment at the 5′ or the 3′ terminus may be determined based on the usage of the microarray, for example, the enzymatic assay downstream of hybridization which is required for analyzing the target nucleic acid. For example, an extension-based SNP microarray requires a free 3′-end, and thus, such microarray comprises oligonucleotide probes each attached to a solid substrate via the 5′ end, leaving the 3′ end free for extension by a polymerase. However, a ligation-based SNP microarray may comprise oligonucleotide probes each attached to a solid substrate either at the 5′ end or at the 3′ end.

The solid substrate can be any suitable material or group of materials having a rigid or semi-rigid surface or surfaces, and the substrate is substantially inert to the various reagents for using or reusing the microarray. In some embodiments, at least one surface of the solid substrate is substantially flat. In some embodiments, the solid substrate takes the form of a bead, resin, gel, microsphere, or other geometric configurations. In some embodiments, the solid substrate is further disposed in a compartment of a solid carrier, such as a multi-well plate, or a fiber optic bundle, or a glass slide. In some embodiments, the solid substrate is suitable for detection of signals, such as fluorescence, from the target nucleic acid hybridized to the oligonucleotide probe. For example, the solid substrate may be optically transparent to allow fluorescence imaging (such as scan or microscopy). In some embodiments, the solid substrate is a glass slide.

The oligonucleotide probe can be attached to the solid substrate covalently or noncovalently so that the oligonucleotide probe remains immobilized during use or reuse of the microarray. Methods of immobilizing oligonucleotides on a solid substrate are known in the art. For example, a silica surface may be first derivatized with a silane reagent containing a functional group, such as sulfhydryl, amine, hydroxyl, or carboxylic acid group, which can be crosslinked to the 5′ terminal nucleotide or the 3′ terminal nucleotide of the oligonucleotide probe via a corresponding reactive functional linker. For example, a sulfhydryl reactive linker may contain a maleimide group. An amine reactive linker may contain a succinimidyl ester (NHS) or isothiocyanate (ITC) group. In some embodiments, the solid surface and the oligonucleotide probe are covalently linked to each other via a bifunctional linker. See, for example, U.S. Pat. No. 5,412,087 and Sheng H. and Ye B C. Appl. Biochem. Biotech. 2009 152(1): 54-56. In some embodiments, the oligonucleotide probe is attached to the solid substrate via noncovalent interactions between two binding moieties.

The oligonucleotide probe may be attached to the solid substrate at a pre-determined position, or at a random position on the solid substrate. In some embodiments, wherein the oligonucleotide probe is attached to a pre-determined position on the solid substrate, the positional information of a signal on the microarray may be used to retrieve the sequence of the oligonucleotide probe that gives rise to the signal. In some embodiments, wherein the oligonucleotide probe is attached to a random position on the solid substrate, the oligonucleotide probe may comprise an address sequence, which allows decoding of the sequence of the oligonucleotide probe. See, for example, Gunderson et al., Genome Research, 14, 870-877, 2004.

### SNP Microarrays

Some embodiments of the present application provide SNP microarrays, which are microarrays having oligonucleotide probes specific for SNP alleles. A “single nucleotide polymorphism” or “SNP” is a locus present in a population which displays a variation in the identity of a single nucleotide between members of the population. A variety of assays have been developed to detect SNPs using a microarray, including hybridization-based assays, and enzyme-based assays, such as extension-based or ligation-based assays. See, for example, Gunderson K L et al. Nature Genetics, 2005, 37:549. The microarrays described in the present application can be compatible with any of the assay formats known in the art. For example, a microarray having the oligonucleotide probes attached to the solid substrate via the 5′ termini while leaving the 3′ termini free in solution, and in which the 3′ terminal nucleotide corresponds to the SNP locus, or 1-2 nucleotides downstream or upstream of the SNP locus, is compatible with extension-based assay for SNP detection.

In some embodiments, there is provided a SNP microarray comprising a plurality of single-stranded oligonucleotide probes each comprising a first terminus and a second terminus, wherein each oligonucleotide probe is attached to a solid substrate via the first terminus, wherein the terminal nucleotide at the second terminus of each oligonucleotide probe is resistant to cleavage, and wherein each oligonucleotide probe has a matching (e.g., perfectly matching) sequence immediately upstream or immediately downstream of a single-nucleotide polymorphism (SNP), and wherein at least two oligonucleotide probes on the microarray are different. In some embodiments, the last two or more (e.g., the last 3, 4, 5 or more) nucleotides at the second terminus of each oligonucleotide probe are resistant to cleavage. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 5′ terminus, wherein the 3′ terminal nucleotide of each oligonucleotide probe is resistant to cleavage (e.g., chemical cleavage, or cleavage by nuclease), and wherein each oligonucleotide probe has a substantially identical (e.g., completely identical) sequence immediately upstream or a substantially complementary (e.g., completely complementary) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 3′ terminus of each oligonucleotide probe are resistant to cleavage (e.g., chemical cleavage, or cleavage by nuclease). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 3′ terminus, wherein the 5′ terminal nucleotide of each oligonucleotide probe is resistant to cleavage (e.g., chemical cleavage, or cleavage by nuclease), and wherein each oligonucleotide probe has a substantially complementary (e.g., completely complementary) sequence immediately upstream or a substantially identical (e.g., completely identical) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 5′ terminus of each oligonucleotide probe are resistant to cleavage (e.g., chemical cleavage, or cleavage by nuclease). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a modified linkage group (e.g., phosphorothioate or phosphoroselenoate), and wherein each oligonucleotide probe has a substantially identical (e.g., completely identical) sequence immediately upstream or a substantially complementary (e.g., completely complementary) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 3′ terminus of each oligonucleotide probe are each linked to their neighboring nucleotides via modified linkage groups (e.g., phosphorothioate or phosphoroselenoate). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a modified linkage group (e.g., phosphorothioate or phosphoroselenoate), and wherein each oligonucleotide probe has a substantially complementary (e.g., completely complementary) sequence immediately upstream or a substantially identical (e.g., completely identical) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 5′ terminus of each oligonucleotide probe are each linked to their neighboring nucleotides via modified linkage groups (e.g., phosphorothioate or phosphoroselenoate). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group (such as the Sp stereoisomer), and wherein each oligonucleotide probe has a substantially identical (e.g., completely identical) sequence immediately upstream or a substantially complementary (e.g., completely complementary) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 3′ terminus of each oligonucleotide probe are each linked to their neighboring nucleotides via phosphorothioate groups (such as the Sp stereoisomer). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a phosphorothioate group (such as the Sp stereoisomer), and wherein each oligonucleotide probe has a substantially complementary (e.g., completely complementary) sequence immediately upstream or a substantially identical (e.g., completely identical) sequence immediately downstream of a single-nucleotide polymorphism (SNP), and wherein at least two oligonucleotide probes on the microarray are different. In some embodiments, the last two or more (e.g., the last 3, 4, 5 or more) nucleotides at the 5′ terminus of each oligonucleotide probe are each linked to their neighboring nucleotides via phosphorothioate groups (such as the Sp stereoisomer). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group in the Sp configuration, wherein the 3′ terminal nucleotide is the only nucleotide in the oligonucleotide probe that is resistant to a 3′ to 5′ exonuclease, and wherein each oligonucleotide probe has a substantially identical (e.g., completely identical) sequence immediately upstream or a substantially complementary (e.g., completely complementary) sequence immediately downstream of a single-nucleotide polymorphism (SNP), and wherein at least two oligonucleotide probes on the microarray are different. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probes, wherein each oligonucleotide probe is attached to a solid substrate via the 3′ terminus, wherein the 5′ terminal nucleotide is linked to the penultimate nucleotide via a phosphorothioate group in the Sp configuration, and wherein the 5′ terminal nucleotide is the only nucleotide in the oligonucleotide probe that is resistant to a 5′ to 3′ exonuclease, and wherein each oligonucleotide probe has a substantially complementary (e.g., completely complementary) sequence immediately upstream or a substantially identical (e.g., completely identical) sequence immediately downstream of a single-nucleotide polymorphism (SNP), and wherein at least two oligonucleotide probes on the microarray are different. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads).

In some embodiments, there is provided a SNP microarray comprising a plurality of single-stranded oligonucleotide probe pairs each comprising a first probe and a second probe, wherein each probe has a first terminus and a second terminus, wherein each probe is attached to a solid substrate via the first terminus, wherein the terminal nucleotide at the second terminus of each probe is resistant to cleavage (e.g., chemical cleavage or cleavage by nuclease), wherein the first probe and the second probe each comprises a matching (e.g., perfectly matching) sequence immediately upstream or immediately downstream of a single-nucleotide polymorphism (SNP), and wherein the terminal nucleotide at the second terminus of the first probe matches a first allele of the SNP and the terminal nucleotide at the second terminus of the second probe matches a second allele of the SNP. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the last two or more (e.g., the last 3, 4, 5 or more) nucleotides at the second terminus of each oligonucleotide probe are resistant to cleavage (e.g., chemical cleavage or cleavage by nuclease). In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 5′ terminus, wherein the 3′ terminal nucleotide of each probe is resistant to cleavage, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP. In some embodiments, the last two or more (e.g., the last 3, 4, 5 or more) nucleotides at the 3′ terminus of each oligonucleotide probe are resistant to cleavage. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 3′ terminus, wherein the 5′ terminal nucleotide of each probe is resistant to cleavage, wherein the first probe and the second probe each comprises a substantially complementary (e.g., completely complementary) sequence immediately upstream or a substantially identical (e.g., completely identical) sequence immediately downstream of a single-nucleotide polymorphism (SNP), and wherein the 5′ terminal nucleotide of the first probe matches a first allele of the SNP and the 5′ terminal nucleotide of the second probe matches a second allele of the SNP. In some embodiments, the last two or more (e.g., the last 3, 4, 5 or more) nucleotides at the 5′ terminus of each oligonucleotide probe are resistant to cleavage. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 5′ terminus, wherein the 3′ terminal nucleotide of each probe is resistant to a nuclease (such as exonuclease), wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP. In some embodiments, the last two or more (e.g., the last 3, 4, 5 or more) nucleotides at the 3′ terminus of each oligonucleotide probe are resistant to the nuclease. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 3′ terminus, wherein the 5′ terminal nucleotide of each probe is resistant to a nuclease (such as exonuclease), wherein the first probe and the second probe each comprises a substantially complementary (e.g., completely complementary) sequence immediately upstream or a substantially identical (e.g., completely identical) sequence immediately downstream of a single-nucleotide polymorphism (SNP), and wherein the 5′ terminal nucleotide of the first probe matches a first allele of the SNP and the 5′ terminal nucleotide of the second probe matches a second allele of the SNP. In some embodiments, the last two or more (e.g., the last 3, 4, 5 or more) nucleotides at the 5′ terminus of each oligonucleotide probe are resistant to the nuclease. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group or phosphoroselenoate group (such as the Sp stereoisomer), wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP. In some embodiments, the last two or more (e.g., the last 3, 4, 5 or more) nucleotides at the 3′ terminus of each oligonucleotide probe are each linked to their neighboring nucleotides via phosphorothioate groups or phosphoroselenoate groups (such as the Sp stereoisomer). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 3′ terminus, wherein the 5′ terminal nucleotide is linked to the penultimate nucleotide via a phosphorothioate or phosphoroselenoate group (such as the Sp stereoisomer), wherein the first probe and the second probe each comprises a substantially complementary (e.g., completely complementary) sequence immediately upstream or a substantially identical (e.g., completely identical) sequence immediately downstream of a single-nucleotide polymorphism (SNP), and wherein the 5′ terminal nucleotide of the first probe matches a first allele of the SNP and the 5′ terminal nucleotide of the second probe matches a second allele of the SNP. In some embodiments, the last two or more (e.g., the last 3, 4, 5 or more) nucleotides at the 5′ terminus of each oligonucleotide probe are each linked to their neighboring nucleotides via phosphorothioate groups or phosphoroselenoate groups (such as the Sp stereoisomer). In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group in the Sp configuration, wherein the 3′ terminal nucleotide is the only nucleotide in each probe that is resistant to a 3′ to 5′ exonuclease, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

In some embodiments, there is provided a SNP microarray comprising a plurality of single-stranded oligonucleotide probe pairs each comprising a first probe and a second probe, wherein each probe is attached to a solid substrate via the 3′ terminus, wherein the 5′ terminal nucleotide is linked to the penultimate nucleotide via a phosphorothioate group in the Sp configuration, wherein the 5′ terminal nucleotide is the only nucleotide in each probe that is resistant to a 5′ to 3′ exonuclease, wherein the first probe and the second probe each comprises a substantially complementary (e.g., completely complementary) sequence immediately upstream or a substantially identical (e.g., completely identical) sequence immediately downstream of a single-nucleotide polymorphism (SNP), and wherein the 5′ terminal nucleotide of the first probe matches a first allele of the SNP and the 5′ terminal nucleotide of the second probe matches a second allele of the SNP. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to a pre-determined position on the solid substrate. In some embodiments, the plurality of oligonucleotide probes is attached to different solid substrates (such as beads). In some embodiments, each oligonucleotide probe pair is attached to the same solid substrate (such as bead).

The sequences of the oligonucleotide probes on the SNP microarray may be designed based on known sequence information of SNPs of interest. Exemplary resources that provide known SNPs include, but are not limited to, the dbSNP administered by the NCBI and available online at ncbi.nlm.nih.gov/SNP/. To detect SNPs using a ligation-based assay on any one of the SNP microarrays described herein, a free adapter oligonucleotide can be designed for each oligonucleotide probe on the SNP microarray so that the free adapter oligonucleotide and the corresponding immobilized oligonucleotide probe on the SNP microarray hybridize to the same strand of a target nucleic acid containing the corresponding SNP of interest, and the free adapter oligonucleotide and the corresponding immobilized oligonucleotide probe flanks the SNP locus. For example, wherein the immobilized oligonucleotide probe on the SNP microarray comprises a substantially identical (e.g., completely identical) sequence immediately upstream of a SNP of interest and a 3′ terminal nucleotide on the free terminus that is identical to a SNP allele, the free adapter oligonucleotide is designed to comprise a substantially identical (e.g., completely identical) sequence immediately downstream the SNP; wherein the immobilized oligonucleotide probe on the SNP microarray comprises a substantially identical (e.g., completely identical) sequence immediately downstream of a SNP of interest and a 5′ terminal nucleotide on the free terminus that is identical to a SNP allele, the free adapter oligonucleotide is designed to comprise a substantially identical (e.g., completely identical) sequence immediately upstream the SNP. Alternatively, a pool of free adapter oligonucleotides having random sequences can be used with the immobilized oligonucleotide probe in a ligation-based SNP detection assay.

An exemplary SNP microarray of the present application has 24-mer single-stranded DNA probes attached to a solid substrate at their 5′ termini, and their 3′ termini are free in solution. The last linkage in these oligonucleotide probes at the 3′ termini is modified to create a phosphorothioate linkage. The phosphorothioate linkage protects the 3′ end of the oligonucleotide probes from degradation by 3′→5′ exonucleases and by polymerases with 3′→5′ proofreading activities. The other linkages in the oligonucleotide probes are all normal phosphodiester linkages, which are susceptible to 3′→5′ exonucleases. The 3′ terminal nucleotide in each probe is designed to be complementary to one allele of a SNP in the target nucleic acid at the same position. 2 probes are created on the microarray for each SNP, one for each allele. Such microarray is compatible with extension based genotyping chemistry or ligation-based genotyping chemistry for SNP detection, and so are afforded the benefits of high accuracy combined with high chip densities, but that can be quickly and cheaply reset to their initial state and used multiple times using a 3′→5′ exonuclease, such as Exo III, Exo T, RecBCD (Exo V), Exo VI, or BAL-31.

### Methods of Preparing Microarray

The present application also provides methods of preparing the immobilized oligonucleotide probes, and the microarrays (including SNP microarrays) described herein.

One approach is to chemically synthesize the oligonucleotide probes comprising the cleavage-resistant terminal nucleotide off the solid substrate by standard oligonucleotide synthesis techniques, and then attached to either a solid substrate or to a bead that was then attached to a solid carrier via the desired terminus. This approach is especially suitable for preparation of bead-based microarrays which have different oligonucleotide probes attached to different beads at random positions which are then distributed on a solid carrier.

Alternatively, the oligonucleotide can be synthesized in situ on the solid substrate, and care is taken to attach the oligonucleotide probe at the desired terminus. For example, an extension-based SNP microarray must have each probe attached to the solid substrate via the 5′ end. During the synthesis of the oligonucleotide probes, a modification may be incorporated in the terminal nucleotide at the free terminus, such as the linkage between the terminal nucleotide and the penultimate nucleotide, to render the terminal nucleotide resistant to cleavage either chemically or enzymatically (e.g., exonuclease).

In some embodiments, there is provided a method of preparing a reusable microarray, comprising: (a) providing a plurality of single-stranded oligonucleotide probes, wherein the 3′ terminal nucleotide of each oligonucleotide probe is resistant to cleavage (such as by a 3′ to 5′ exonuclease), and wherein at least two of the oligonucleotide probes on the microarray are different; and (b) attaching the 5′ terminus of each oligonucleotide probe to a solid substrate, thereby providing the reusable microarray. In some embodiments, the last two or more (e.g., the last 3, 4, 5 or more) nucleotides at the second terminus of each oligonucleotide probe are resistant to cleavage. In some embodiments, the 3′ terminal nucleotide is linked to the penultimate nucleotide via a phosphorothioate or phosphoroselenoate group. In some embodiments, the last two or more (e.g., the last 3, 4, 5 or more) nucleotides at the 3′ terminus are each linked to their neighboring nucleotides via phosphorothioate or phosphoroselenoate groups. In some embodiments, the phosphorothioate or phosphoroselenoate group is in the Sp configuration. In some embodiments, the oligonucleotide probe is stereoselectively synthesized to have the phosphorothioate or phosphoroselenoate group(s) in the Sp configuration. In some embodiments, the method further comprises treating the reusable microarray with a 3′ to 5′ exonuclease. In some embodiments, the plurality of oligonucleotide probes comprises at least about 1000 (e.g., at least about any of 10⁴, 10⁵, 10⁶, or more) different oligonucleotide probes. In some embodiments, the oligonucleotide probes are attached to different solid substrates (such as beads). In some embodiments, the solid substrates are further attached to a solid carrier (such as glass slide or fiber optic bundle).

In some embodiments, there is provided a method of preparing a reusable microarray, comprising: (a) providing a plurality of single-stranded oligonucleotides, wherein the 5′ terminal nucleotide of each oligonucleotide is resistant to cleavage (such as by a 5′ to 3′ exonuclease), and wherein at least two of the single-stranded oligonucleotides are different; and (b) attaching the 3′ terminus of each single-stranded oligonucleotide to a solid substrate, thereby providing the reusable microarray. In some embodiments, the 5′ terminal nucleotide is linked to the penultimate nucleotide via a phosphorothioate or phosphoroselenoate group. In some embodiments, the last two or more (e.g., the last 3, 4, 5 or more) nucleotides at the 5′ terminus are each linked to their neighboring nucleotides via phosphorothioate or phosphoroselenoate groups. In some embodiments, the phosphorothioate or phosphoroselenoate group is in the Sp configuration. In some embodiments, the oligonucleotide probe is stereoselectively synthesized to have the phosphorothioate or phosphoroselenoate group(s) in the Sp configuration. In some embodiments, the method further comprises treating the reusable microarray with a 5′ to 3′ exonuclease. In some embodiments, the plurality of oligonucleotide probes comprises at least about 1000 (e.g., at least about any of 10⁴, 10⁵, 10⁶, or more) different oligonucleotide probes. In some embodiments, the oligonucleotide probes are attached to different solid substrates (such as beads). In some embodiments, the solid substrates are further attached to a solid carrier (such as glass slide or fiber optic bundle).

In some embodiments, there is provided a method of preparing a reusable microarray, comprising: synthesizing a plurality of single-stranded oligonucleotide probes each having a first terminus and a second terminus on a solid substrate, wherein the first terminus of each oligonucleotide probe is attached to the solid substrate, and wherein the terminal nucleotide at the second terminus of each oligonucleotide probe is resistant to cleavage, and wherein at least two of the oligonucleotide probes on the microarray are different, thereby providing the reusable microarray. In some embodiments, the last two or more (e.g., the last 3, 4, 5 or more) nucleotides at the second terminus of each oligonucleotide probe are resistant to cleavage. In some embodiments, the plurality of oligonucleotide probes comprises at least about 1000 (e.g., at least about any of 10⁴, 10⁵, 10⁶, or more) different oligonucleotide probes. In some embodiments, each oligonucleotide probe is synthesized at a pre-determined position on the solid substrate. In some embodiments, the solid substrate is a glass slide.

In some embodiments, there is provided a method of preparing a reusable microarray, comprising: synthesizing a plurality of single-stranded oligonucleotide probes on a solid substrate, wherein each oligonucleotide probe is attached to the solid substrate via the 5′ terminus, and wherein the 3′ terminal nucleotide of each oligonucleotide probe is resistant to cleavage (such as by a 3′ to 5′ exonuclease), and wherein at least two of the oligonucleotide probes on the microarray are different, thereby providing the reusable microarray. In some embodiments, the last two or more (e.g., the last 3, 4, 5 or more) nucleotides at the 3′ terminus of each oligonucleotide probe are resistant to cleavage. In some embodiments, the method further comprises treating the reusable microarray with a cleavage method (such as a 3′ to 5′ exonuclease). In some embodiments, the plurality of oligonucleotide probes comprises at least about 1000 at least (e.g., about any of 10⁴, 10⁵, 10⁶, or more) different oligonucleotide probes. In some embodiments, each oligonucleotide probe is synthesized at a pre-determined position on the solid substrate. In some embodiments, the solid substrate is a glass slide.

In some embodiments, there is provided a method of preparing a reusable microarray, comprising: synthesizing a plurality of single-stranded oligonucleotide probes on a solid substrate, wherein each oligonucleotide probe is attached to the solid substrate via the 3′ terminus, and wherein the 5′ terminal nucleotide of each oligonucleotide probe is resistant to cleavage (such as by a 3′ to 5′ exonuclease), and wherein at least two of the oligonucleotide probes on the microarray are different, thereby providing the reusable microarray. In some embodiments, the last two or more (e.g., the last 3, 4, 5 or more) nucleotides at the 5′ terminus of each oligonucleotide probe are resistant to cleavage. In some embodiments, the method further comprises treating the reusable microarray with a cleavage method (such as a 5′ to 3′ exonuclease). In some embodiments, the plurality of oligonucleotide probes comprises at least about 1000 (e.g., at least about any of 10⁴, 10⁵, 10⁶, or more) different oligonucleotide probes. In some embodiments, each oligonucleotide probe is synthesized at a pre-determined position on the solid substrate. In some embodiments, the solid substrate is a glass slide.

In some embodiments, there is provided a method of preparing a reusable microarray, comprising: (a) synthesizing a plurality of single-stranded oligonucleotide probes (such as DNA probes) on a solid substrate, wherein each oligonucleotide probe is attached to the solid substrate via the 5′ terminus, wherein the 3′ terminal nucleotide is linked to the penultimate nucleotide via a phosphorothioate or phosphoroselenoate group, and wherein at least two of the oligonucleotide probes on the microarray are different; and (b) treating the plurality of oligonucleotide probes with a 3′ to 5′ exonuclease, thereby providing the reusable microarray. In some embodiments, the plurality of oligonucleotide probes comprises at least about 1000 (e.g., at least about any of 10⁴, 10⁵, 10⁶, or more) different oligonucleotide probes. In some embodiments, the last two or more (e.g., the last 3, 4, 5 or more) nucleotides at the 3′ terminus are each linked to their neighboring nucleotides via phosphorothioate or phosphoroselenoate groups. In some embodiments, the oligonucleotide probes are synthesized using phosphoramidite chemistry. In some embodiments, the oligonucleotide probes are synthesized from 3′ to 5′ and then flipped on the solid substrate to attach the 5′ termini to the solid substrate. In some embodiments, the phosphorothioate linkage is synthesized by sulfurization of a phosphite triester linkage. In some embodiments, each oligonucleotide probe is synthesized at a pre-determined position on the solid substrate. In some embodiments, the solid substrate is a glass slide.

In some embodiments, there is provided a method of preparing a reusable microarray, comprising: (a) synthesizing a plurality of single-stranded oligonucleotide probes (such as DNA probes) on a solid substrate, wherein each oligonucleotide probe is attached to the solid substrate via the 3′ terminus, wherein the 5′ terminal nucleotide is linked to the penultimate nucleotide via a phosphorothioate or phosphoroselenoate group, and wherein at least two of the oligonucleotide probes on the microarray are different; and (b) treating the plurality of oligonucleotide probes with a 5′ to 3′ exonuclease, thereby providing the reusable microarray. In some embodiments, the last two or more (e.g., the last 3, 4, 5 or more) nucleotides at the 5′ terminus are each linked to their neighboring nucleotides via phosphorothioate or phosphoroselenoate groups. In some embodiments, the oligonucleotide probes are synthesized using phosphoramidite chemistry. In some embodiments, the phosphorothioate linkage is synthesized by sulfurization of a phosphite triester linkage. In some embodiments, the plurality of oligonucleotide probes comprises at least about 1000 (e.g., at least about any of 10⁴, 10⁵, 10⁶, or more) different oligonucleotide probes. In some embodiments, each oligonucleotide probe is synthesized at a pre-determined position on the solid substrate. In some embodiments, the solid substrate is a glass slide.

Further provided are reusable microarrays prepared by any one of the methods of preparing described herein.

The oligonucleotide probes can be synthesized using any suitable oligonucleotide synthesis methods known in the art, including automated phosphoramidite chemistry. For example, light-directed combinatorial synthesis of oligonucleotide arrays on a solid substrate (such as glass surface) are also known in the art using either mask-guided or maskless methods. See, for example, Fodor S P et al. Science. 1991 Feb. 15; 251(4995):767-73; and Nuwaysir E F et al. Genome Res. 2002 November; 12(11):1749-55. Briefly, the solid substrate is derivatized with functional groups blocked by a photolabile protection group to allow photolysis through a photolithographic mask or using micro-mirrors to selectively expose the functional groups which are then ready to react with incoming 5′ or 3′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents. In some embodiments, inkjet printing technology may be used in combination with standard oligonucleotide synthesis chemistry to produce oligonucleotide arrays in situ. See, for example, Blanchard A P, et al. Biosensor and Bioelectronics. 1996; 11:687-690. In some embodiments, the oligonucleotide probes are synthesized on beads, and the beads are then randomly assembled on a solid carrier, such as fiber optic bundle or glass slide. See, for example, Walt D R Science. 2000 Jan. 21; 287(5452):451-2.

Phosphorothioate linkages can be introduced to oligonucleotide probes during synthesis using phosphoramidite, in which following coupling of a phosphoramidite containing nucleotide to the growing oligonucleotide chain, a phosphite triester linkage is formed between the incoming nucleotide and the existent oligonucleotide chain. To obtain a phosphorothioate linkage, the oligonucleotide is subject to a sulfurizing agent, which converts the phosphite triester linkage to a phosphorothioate group. This sulfurization step is usually done when the backbone linkage of the DNA to be sulfurized has not yet been oxidized, and is still in the form of a phosphite linkage (phosphoramidite), which is much more susceptible to sulfurization, and as such the phosphorus is sulfurized instead of being oxidized as in the standard phosphoramidite oligonucleotide synthesis chemistry. Suitable sulfurizing agents that can be used to introduce the phosphorothioate group either in situ or off the chip include, but are not limited to, 3-(Dimethylaminomethylidene)amino-3H-1,2,4-dithiazole-3-thione (DDTT), 3H-1,2-benzodithiol-3-one 1,1-dioxide (Beaucage reagent), N,N,N′,N′-Tetraethylthiuram disulfide (TETD) and phenylacetyl disulfide (PADS). In the cases where only the terminal nucleotide at the second terminus has the phosphorothioate linkage, prior to addition of the terminal nucleotide, the growing oligonucleotides are fully oxidized to ensure that no linkages other than the linkage between the terminal nucleotide and the penultimate nucleotide at the second terminus are phosphorothioate groups, which are resistant to exonucleases.

In some embodiments, only a stereochemically pure species of the oligonucleotide probes with respect to the modification to the terminal nucleotide at the second terminus is resistant to the cleavage method, especially for nucleases, such as exonucleases, but chemical synthesis of the oligonucleotide probes in situ or off the solid substrate normally give rise to a racemic mixture of oligonucleotide probes. The non-cleavage resistant oligonucleotide probe may give rise to noise due to partial degradation and nonspecific binding to target nucleic acids when the microarray is used. For example, standard phosphoramidite synthesis of oligonucleotides using a sulfurization step as described above normally provide a random 50:50 mixture of the phosphorothioate group, consisting of the exonuclease-sensitive Rp stereoisomer, and the exonuclease-resistant Sp stereoisomer. Thus, in some embodiments, the synthesized oligonucleotide probes are treated with the cleavage method (such as exonuclease) under a condition that removes probes containing the non-cleavage resistant species. Such treatment also provides the benefit of removing truncated or partial oligonucleotide products due to failed synthesis steps, thereby further reducing noise due to nonspecific binding to the target nucleic acids. In some embodiments, the synthesized oligonucleotide probes are treated with an exonuclease for about any one of 30 minutes, 1 hour, 2 hours, 3 hours, 6 hours, 12 hours, or more. The exonuclease used can be the same exonuclease that is used to regenerate the microarray after use. In some embodiments, the oligonucleotide probe is stereoselectively synthesized to have the phosphorothioate group(s) in the Sp configuration. See, for example, Guga P. and Stec W J. Current Protocols in Nucleic Acid Chemistry, 14:4.17:4.17.1-4.17.28 (2003).

### METHODS OF USE

The oligonucleotide probes and microarrays describe in the present application may be used in a variety of applications, including gene expression analysis and genotyping in clinical diagnosis, agricultural, environmental, and forensic settings. The microarrays described herein are especially useful for detection of single-nucleotide polymorphisms using enzyme-based assays, such as extension-based assays or ligation-based assays. The present application further provides methods of reusing the microarrays, thereby reducing the cost of detection per sample.

In some embodiments, there is provided a method of detecting one or more alleles in a sample of target nucleic acids, comprising: (a) hybridizing the sample of target nucleic acids to any one of the oligonucleotide probes described herein, or any one of the microarrays described herein to provide probe-target hybrids; and (b) detecting the probe-target hybrids, thereby detecting the one or more alleles. In some embodiments, the method further comprises: (c) treating the oligonucleotide probe or the microarray with a cleavage method, such as a chemical cleavage method or an exonuclease. In some embodiments, the treating step comprises subjecting the oligonucleotide probe or the microarray to a denaturing condition, such as chemical denaturation and/or heat denaturation. In some embodiments, the method further comprises: (d) reusing the oligonucleotide probe or the microarray according to steps (a) and (b). In some embodiments, the oligonucleotide probe or the microarray is reused for about 2 times to about 100 times.

In some embodiments, there is provided a method of detecting the presence or absence of a single-nucleotide polymorphism (SNP) allele in a sample of target nucleic acids, comprising: (a) hybridizing the sample to the oligonucleotide probes of any one of the SNP microarrays described herein to provide probe-target hybrids, wherein the first terminus is the 5′ terminus, and wherein at least one oligonucleotide probe pair comprises a sequence that matches (e.g., perfectly matches) the SNP allele; (b) contacting the probe-target hybrids with a polymerase and nucleotides under a condition that allows primer extension to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the polymerase is a polymerase with proofreading activity. In some embodiments, the method further comprises: (d) treating the oligonucleotide probe or the microarray with a cleavage method, e.g., a chemical cleavage method or an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group (such as the Sp stereoisomer), and wherein each oligonucleotide probe has a substantially identical (e.g., identical) sequence immediately upstream or a substantially complementary (e.g., completely complementary) sequence immediately downstream of the SNP, and wherein at least two oligonucleotide probes on the microarray are different; (b) contacting the probe-target hybrids with a polymerase and chain-terminating nucleotides comprising at least two different labels under a condition that allows single base extension to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, A and G chain terminating nucleotides are labelled with a first hapten, and C and T chain-terminating nucleotides are labelled with a second hapten. In some embodiments, the chain-terminating nucleotides are dideoxyribonucleotides. In some embodiments, step (c) comprises contacting the modified probe-target hybrids with a first protein (e.g., antibody) that specifically binds to the first hapten and a second protein (e.g., antibody) that specifically binds to the second hapten, and detecting the first protein and the second protein attached to the modified probe-target hybrids. In some embodiments, the first protein is labelled with a first fluorophore and the second protein is labelled with a second fluorophore. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 3.

In the example of FIG. 3, the oligonucleotide probes are designed to stop just short of SNP (either immediately downstream or immediately upstream) and extension is carried out with chain terminating dideoxyribonucleotides labelled with 2 different haptens. The polymerase used in the extension is able to discriminate between the 2 alleles of the SNP and the result is read out using 2 fluorescently labelled antibodies against the 2 haptens. The fluorescence must be measured in 2 channels to measure both fluorophores. This assay requires only a single oligonucleotide probe per SNP.

In some embodiments, there is provided a method of detecting the presence or absence of a single-nucleotide polymorphism (SNP) allele in a sample of target nucleic acids, comprising: (a) hybridizing the sample to the oligonucleotide probes of the SNP microarrays described herein in the presence of a free adapter oligonucleotide to provide probe-target hybrids, wherein at least one oligonucleotide probe pair comprises a sequence that matches (e.g., perfectly matches) the SNP allele; (b) contacting the probe-target hybrids with a ligase in the presence of a plurality of free oligonucleotides under a condition that that allows allele-specific ligation to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, each free oligonucleotide is labelled with a fluorophore or a hapten. In some embodiments, the method further comprises: (d) treating the oligonucleotide probe or the microarray with a cleavage method, such as a chemical cleavage method or an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probes, wherein each oligonucleotide probe is attached to a solid substrate via one of the 3′ terminus, wherein the 5′ terminal nucleotide is linked to the penultimate nucleotide via a phosphorothioate group (such as the Sp stereoisomer), and wherein each oligonucleotide probe has a substantially identical (e.g., identical) sequence immediately upstream or a substantially complementary (e.g., completely complementary) sequence immediately downstream of the SNP, and wherein at least two oligonucleotide probes on the microarray are different; (b) contacting the probe-target hybrids with a ligase in the presence of a first pool of free adapter oligonucleotides each labelled with a first hapten or a first fluorophore and a second pool of free adapter oligonucleotides each labelled with a second hapten or a second fluorophore under a condition that allows allele-specific ligation to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the phosphorothioate group has Sp configuration. In some embodiments, the 5′ terminal nucleotide is the only cleavage-resistant nucleotide in each oligonucleotide probe. In some embodiments, the last two or more (e.g., the last 3, 4, 5 or more) nucleotides at the 5′ terminus are each linked to their neighboring nucleotides via phosphorothioate groups (such as the Sp stereoisomer). In some embodiments, the free adapter oligonucleotides comprise random sequences, and the 3′ terminal nucleotide of each free adapter oligonucleotides in the first pool matches the SNP allele, and the 3′ terminal nucleotide of each free adapter oligonucleotide in the second pool is complementary to the 3′ terminal nucleotide of the first pool. In some embodiments, step (c) comprises contacting the modified probe-target hybrids with a first protein (e.g., antibody) that specifically binds to the first hapten and a second protein (e.g., antibody) that specifically binds to the second hapten, and detecting the first protein and the second protein attached to the modified probe-target hybrids. In some embodiments, the first protein is labelled with a first fluorophore and the second protein is labelled with a second fluorophore. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 6.

In the example of FIG. 6, the oligonucleotide probes are attached to the substrate by their 3′ end with the 5′ end free in solution. The 5′ terminus of the oligonucleotide probe is immediately upstream or immediately downstream of the SNP nucleotide. 2 types of free adapter oligonucleotide are used where the 3′ terminal nucleotide matches one allele of the SNP and a different hapten corresponds to a different allele of the SNP (a different nucleotide in the 3′ terminal position). Ligation of these adapters introduces a different hapten depending on the genotype of the SNP. The signal is amplified and measured using 2 different fluorescently labelled antibodies against the haptens. This assay requires only a single oligonucleotide probe per SNP.

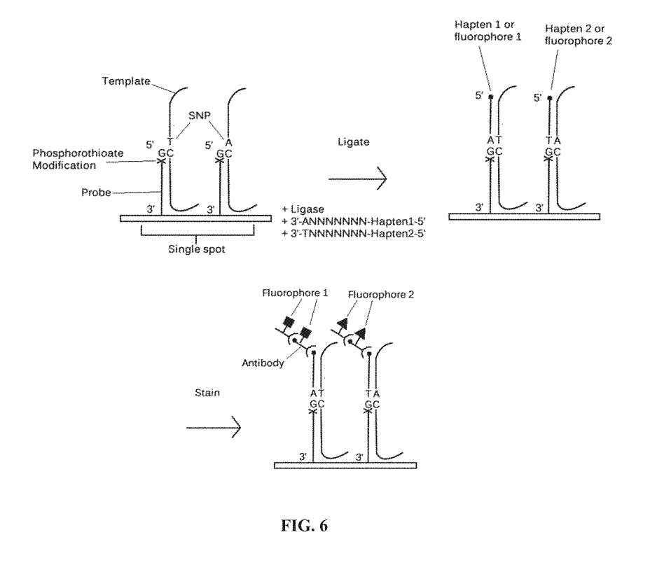

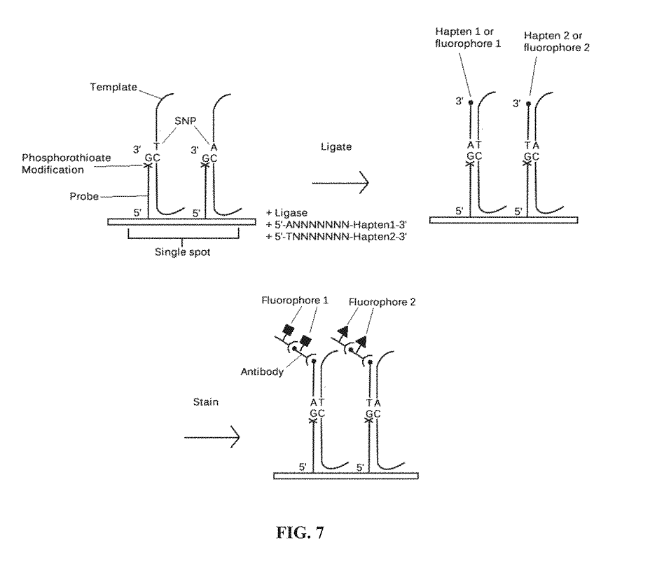

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group (such as the Sp stereoisomer), and wherein each oligonucleotide probe has a substantially identical (e.g., completely identical) sequence immediately upstream or a substantially complementary (e.g., completely complementary) sequence immediately downstream of the SNP, and wherein at least two oligonucleotide probes on the microarray are different; (b) contacting the probe-target hybrids with a ligase in the presence of a first pool of free adapter oligonucleotides each labelled with a first hapten or a first fluorophore and a second pool of free adapter oligonucleotides each labelled with a second hapten or a second fluorophore under a condition that allows allele-specific ligation to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the free adapter oligonucleotides comprise random sequences, and the 5′ terminal nucleotide of each free adapter oligonucleotides in the first pool matches the SNP allele, and the 5′ terminal nucleotide of each free adapter oligonucleotide in the second pool is complementary to the 5′ terminal nucleotide of the first pool. In some embodiments, step (c) comprises contacting the modified probe-target hybrids with a first protein (e.g., antibody) that specifically binds to the first hapten and a second protein (e.g., antibody) that specifically binds to the second hapten, and detecting the first protein and the second protein attached to the modified probe-target hybrids. In some embodiments, the first protein is labelled with a first fluorophore and the second protein is labelled with a second fluorophore. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 7, which is similar to the example shown in FIG. 6, but the oligonucleotide probe has its 5′ terminus attached to the substrate and its 3′ terminus free. The free adapter oligonucleotides are also reversed with respect to those in FIG. 6 with the nucleotide corresponding to the SNP at the 5′ terminus and the hapten at the 3′ terminus.

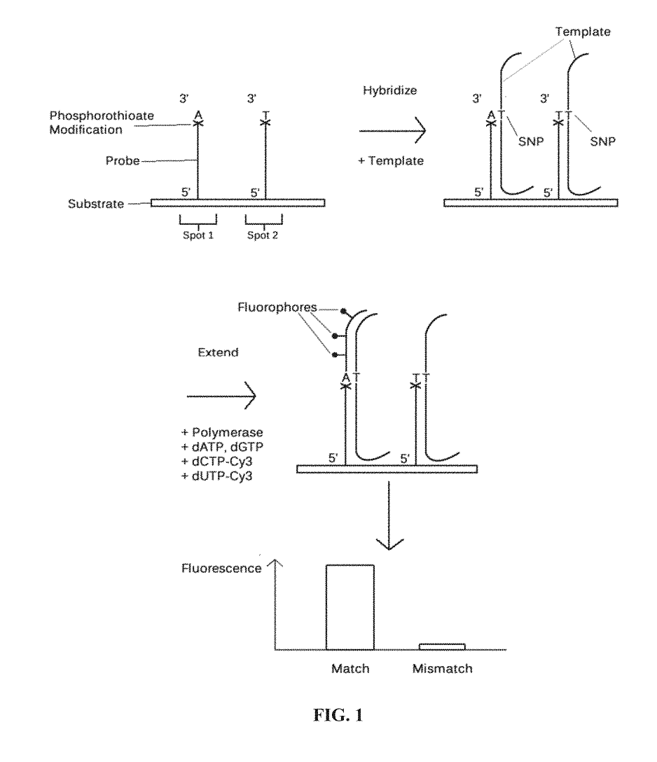

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase and fluorescently labelled nucleotides (e.g., Cy3 labelled) under a condition that allows allele-specific primer extension to provide modified probe-target hybrids; and (c) detecting fluorescence from the nucleotides incorporated in the modified probe-target thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the polymerase is a polymerase with proofreading activity. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 1.

In the example of FIG. 1, oligonucleotide probes of 24 nucleotides in length are attached to a solid substrate by their 5′ termini leaving their 3′ termini free. Target DNA is hybridized to these oligonucleotide probes and extended with a mix of 2 natural and 2 fluorescently labelled nucleotides. The oligonucleotide probe only becomes extended if the last nucleotide of the probe matches the specific allele of the SNP designed to target. This assay uses 2 spots on the microarray chip per SNP with the first 23 bp being identical and the last nucleotide being complementary to the SNP nucleotide in the template. This reaction is allele specific extension (ASPE) based and uses the fact that multiple nucleotides can be incorporated, half of which produce a fluorescent signal, to achieve signal amplification instead of relying on antibody based signal amplification in methods that hapten-labelled nucleotides or adaptor oligonucleotides.

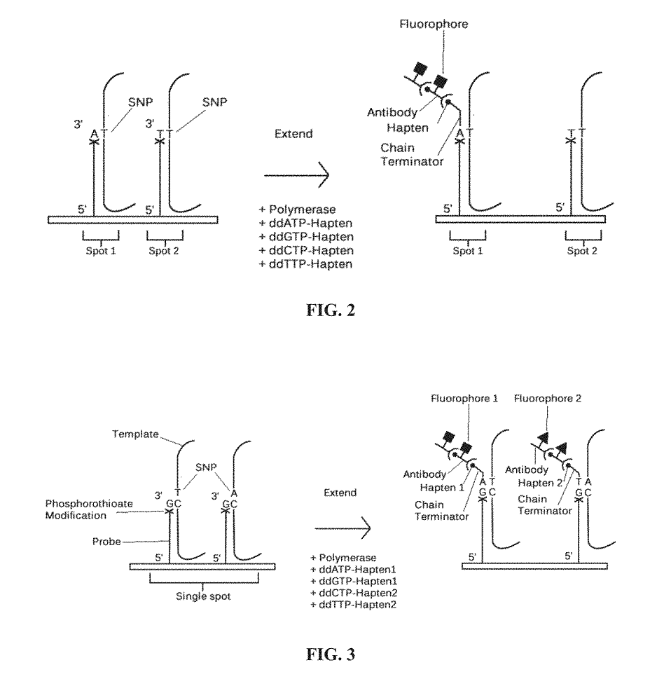

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase and chain-terminating nucleotides comprising at least two different labels under a condition that allows single base extension to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the chain-terminating nucleotides comprise a hapten. In some embodiments, the chain-terminating nucleotides are dideoxyribonucleotides. In some embodiments, step (c) comprises contacting the modified probe-target hybrids with a fluorescently labelled protein (e.g., antibody) that specifically binds to the hapten, and detecting the fluorescently labelled protein attached to the modified probe-target hybrids. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 2.

In the example of FIG. 2, as chain terminating dideoxyribonucleotides are used in the extension step, only a single nucleotide is incorporated. The nucleotides are all labelled with the same hapten as this is a single color assay. Signal amplification is achieved using a fluorescently labelled antibody that binds to the hapten.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase and nucleotides under a condition that allows allele-specific primer extension to provide modified probe-target hybrids; (c) contacting the modified probe-target hybrids with a dye that specifically binds to double-stranded nucleic acids and detecting signal from the dye that is bound to the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the polymerase is a polymerase with proofreading activity. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 8.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase and nucleotides under a condition that allows allele-specific primer extension to provide modified probe-target hybrids; (c) subjecting the modified probe-target hybrids to a denaturing condition to provide modified probes, contacting the modified probes with a dye that specifically binds to single-stranded nucleic acids, and detecting signal from the dye that is bound to the modified probes thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the polymerase is a polymerase with proofreading activity. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 8.

In the example of FIG. 8, the oligonucleotide probes are extended using a proofreading polymerase and natural nucleotides. Following extension, in some embodiments, the target nucleic acid is removed using chemical and/or thermal denaturation and washing, a single-stranded DNA specific dye is then used to measure the amount of DNA in each test area (also referred to herein as “spot”). Spots in which the probe has extended produce significantly more fluorescence and if this fluorescence is measured to be above a threshold, the SNP is called as that particular allele. In some embodiments, the extended probe-target hybrid is not denatured and a double-strand specific DNA binding dye is used to measure the difference in the amount of double stranded DNA between spots where the probes have extended and where they have not. A fluorescence threshold is used to determine which SNP allele to call.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group in the Sp configuration, wherein the 3′ terminal nucleotide is the only nucleotide in each probe that is resistant to a 3′ to 5′ exonuclease, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase and nucleotides under a condition that allows primer extension to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the polymerase is a polymerase with proofreading activity. In some embodiments, the primer extension is allele-specific primer extension (ASPE). In some embodiments, the nucleotides are fluorescently labelled, and wherein the modified probe-target hybrids are detected by detecting fluorescence from the nucleotides incorporated in the modified probe-target hybrids. In some embodiments, step (c) comprises contacting the modified probe-target hybrids with a dye that specifically binds to double-stranded nucleic acids and detecting signal from the dye that is bound to the modified probe-target hybrids. In some embodiments, step (c) comprises subjecting the modified probe-target hybrids to a denaturing condition to provide modified probes, contacting the modified probes with a dye that specifically binds to single-stranded nucleic acids, and detecting signal from the dye that is bound to the modified probes. In some embodiments, the primer extension is single base extension (SBE). In some embodiments, the nucleotides are chain-terminating nucleotides comprising a hapten. In some embodiments, step (c) comprises contacting the modified probe-target hybrids with a fluorescently labelled protein (e.g., antibody) that specifically binds to the hapten, and detecting the fluorescently labelled protein (e.g., antibody) attached to the modified probe-target hybrids. In some embodiments, the method further comprises: (d) treating the oligonucleotide probe or the microarray with the 3′ to 5′ exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times.

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group in the Sp configuration, wherein the 3′ terminal nucleotide is the only nucleotide in each probe that is resistant to a 3′ to 5′ exonuclease, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a polymerase with proofreading activity and fluorescently labelled (such as Cy3 labelled) nucleotides under a condition that allows allele-specific primer extension to provide modified probe-target hybrids; and (c) detecting fluorescence from the nucleotides incorporated in the modified probe-target hybrids, thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the method further comprises: (d) treating the oligonucleotide probe or the microarray with the 3′ to 5′ exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times.

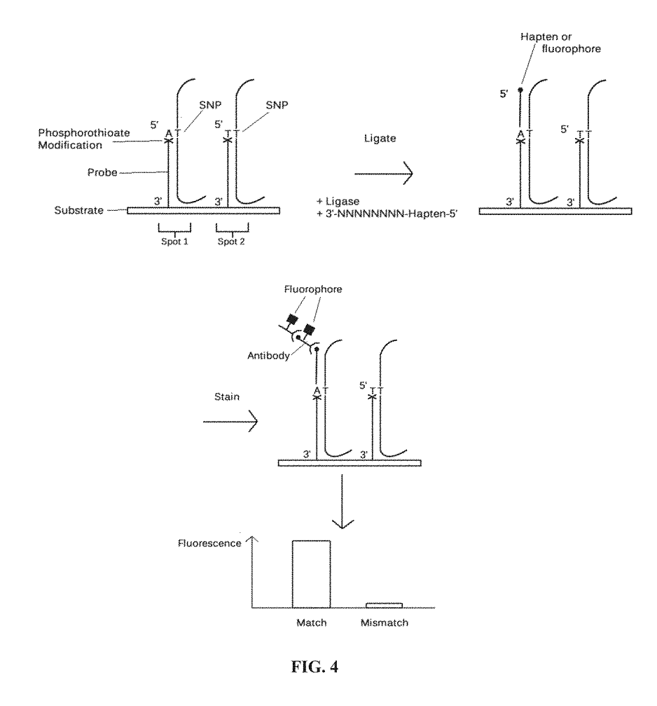

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 3′ terminus, wherein the 5′ terminal nucleotide is linked to the penultimate nucleotide via a phosphorothioate group, wherein the first probe and the second probe each comprises a substantially complementary (e.g., completely complementary) sequence immediately upstream or a substantially identical (e.g., completely identical) sequence immediately downstream of a single-nucleotide polymorphism (SNP), and wherein the 5′ terminal nucleotide of the first probe matches a first allele of the SNP and the 5′ terminal nucleotide of the second probe matches a second allele of the SNP; (b) contacting the probe-target hybrids with a ligase in the presence of a plurality of free adapter oligonucleotides under a condition that allows allele-specific ligation to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the phosphorothioate group has Sp configuration. In some embodiments, the 5′ terminal nucleotide is the only cleavage-resistant nucleotide in each oligonucleotide probe. In some embodiments, the last two or more (e.g., the last 3, 4, 5 or more) nucleotides at the 5′ terminus are each linked to their neighboring nucleotides via phosphorothioate groups (such as the Sp stereoisomer). In some embodiments, the free adapter oligonucleotides comprise random sequences, and the 5′ termini are labelled with a hapten or a fluorophore. In some embodiments, step (c) comprises contacting the modified probe-target hybrids with a fluorescently labelled protein (e.g., antibody) that specifically binds to the hapten, and detecting the fluorescently labelled protein attached to the modified probe-target hybrids. In some embodiments, the method further comprises: (d) treating the oligonucleotide probe or the microarray with an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 4.

In the example of FIG. 4, a free adapter oligonucleotide is added, which contains a mix of random nucleotides between 5 and 50 bp in length with a hapten on the 5′ end. Ligation only occurs if the SNP nucleotide at the 5′ terminus of the probe and the corresponding nucleotide in the target correctly base pair and this base pairing is discriminated by the ligase. If ligation occurs, after the free adapter oligonucleotide has been washed away, the hapten is incorporated into the end of the probe and amplification and detection are achieved using a fluorescently labelled antibody.

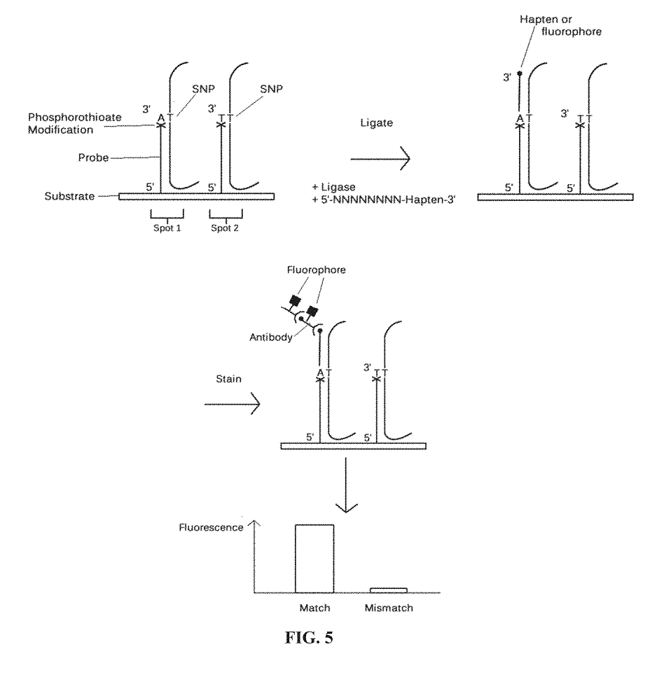

In some embodiments, there is provided a method of detecting the presence or absence of a single-nucleotide polymorphism (SNP) allele in a sample of target nucleic acids, comprising: (a) hybridizing the sample to the oligonucleotide probes of a SNP microarray comprising a plurality of single-stranded oligonucleotide probe pairs each comprising a first probe and a second probe, wherein each probe is attached to a solid substrate via the 5′ terminus, wherein the 3′ terminal nucleotide is linked to the penultimate nucleotide via a phosphorothioate group, wherein the first probe and the second probe each comprises a substantially identical (e.g., completely identical) sequence immediately upstream or a substantially complementary (e.g., completely complementary) sequence immediately downstream of a single-nucleotide polymorphism (SNP), and wherein the 3′ terminal nucleotide of the first probe matches a first allele of the SNP and the 3′ terminal nucleotide of the second probe matches a second allele of the SNP; (b) contacting the probe-target hybrids with a ligase in the presence of a plurality of free adapter oligonucleotides under a condition that allows allele-specific ligation to provide modified probe-target hybrids; and (c) detecting the modified probe-target hybrids thereby detecting the presence or absence of the SNP allele in the target nucleic acid. In some embodiments, the SNP microarray comprises at least about 200 (such as at least about any of 400, 600, 800, 1000, 10⁴, 10⁵, 10⁶, or more) oligonucleotide probes each specific for a different SNP locus of interest. In some embodiments, the phosphorothioate group has Sp configuration. In some embodiments, the 3′ terminal nucleotide is the only cleavage-resistant nucleotide in each oligonucleotide probe. In some embodiments, the last two or more (e.g., the last 3, 4, 5 or more) nucleotides at the 3′ terminus are each linked to their neighboring nucleotides via phosphorothioate groups (such as the Sp stereoisomer). In some embodiments, the free adapter oligonucleotides comprise random sequences, and the 3′ termini are labelled with a hapten or a fluorophore. In some embodiments, step (c) comprises contacting the modified probe-target hybrids with a fluorescently labelled protein (e.g., antibody) that specifically binds to the hapten, and detecting the fluorescently labelled protein attached to the modified probe-target hybrids. In some embodiments, the method further comprises: (d) treating the oligonucleotide probe or the microarray with a cleavage method, such as an exonuclease. In some embodiments, the treating step comprises subjecting the oligonucleotide probe or the microarray to a denaturing condition, such as chemical denaturation and/or heat denaturation. In some embodiments, the method further comprises: (e) reusing the oligonucleotide probe or the microarray according to steps (a)-(c). In some embodiments, the oligonucleotide probe or the microarray is reused for about 2 times to about 100 times. An example is shown in FIG. 5, which is similar to the example of FIG. 4, but the oligonucleotides probe are attached to the solid substrate by their 5′ termini, and the free adapter oligonucleotides have the hapten on the 3′ termini.

The SNP microarrays described herein can be reused, and the SNP detection methods can be repeated. FIG. 9 shows an exemplary schematic of using and reusing any of the SNP microarrays described herein. In the example of FIG. 9, after hybridization of the target nucleic acids to the oligonucleotide probes, either an extension of a ligation reaction is carried out, which covalently modifies the oligonucleotide probes. After detection and SNP calling, the target nucleic acids are removed using chemical and/or heat denaturation and washing. The probes are then contacted with an exonuclease (3′->5′ in this specific example), which removes nucleotides from either the extension or ligation products at the free termini of the oligonucleotide probes until the exonuclease reaches the phosphorothioate modification, which is resistant to cleavage and at this point the exonuclease stops. After a final wash to remove left over exonuclease and cleaved nucleotides, the microarray has been reset and can be used again in the same way.

The methods described herein may be used to analyze any sample of target nucleic acids from any source. The term “target nucleic acid” refers to a nucleic acid molecule which contains a sequence which has at least partial complementarity with at least an oligonucleotide probe. The target nucleic acid may comprise single- or double-stranded DNA or RNA. The term “sample” is used in its broadest sense to include any specimen or culture (e.g., microbiological cultures), as well as biological and environmental samples. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as cows, horses, fish, rodents, etc. Environmental samples include environmental material such as surface matter, soil, water and industrial samples, as well as samples obtained from food and dairy processing instruments, apparatus, equipment, utensils, disposable and non-disposable items.

In some embodiments, the sample of target nucleic acids comprise genomic DNA or genomic DNA fragments. Genomic DNA can be isolated from one or more cells, bodily fluids or tissues. Known methods can be used to obtain a bodily fluid such as blood, sweat, tears, lymph, urine, saliva, semen, cerebrospinal fluid, feces or amniotic fluid. Similarly known biopsy methods can be used to obtain cells or tissues such as buccal swab, mouthwash, surgical removal, biopsy aspiration or the like. Genomic DNA can also be obtained from one or more cell or tissue in primary culture, in a propagated cell line, a fixed archival sample, forensic sample or archeological sample. A genome fragment can be DNA, RNA, or an analog thereof. In some embodiments, the sample of target nucleic acids comprise cDNA or cDNA fragments. cDNAs may be prepared using any known methods in the art, including, for example, reverse transcription from total RNA.

In some embodiments, the sample of target nucleic acids are further amplified to provide nucleic acid fragments prior to hybridization to the microarray. In some embodiments, the amplification is whole genome amplification. In some embodiments, the amplification is targeted amplification that enhance the presentation of certain alleles and loci of interest in the sample to be hybridized to the microarray. In some embodiments, the target nucleic acids are at least about any of 25, 50, 70, 100, 200, 300, 400, 500, 600, 700, 800, 900 or 1000 or more nucleotides long.

The sample of the target nucleic acids is hybridized to the oligonucleotide probes or the microarrays. Depending on the application, complexity of the sample, and the multiplexity (i.e., number of different oligonucleotide probes) on the microarray, an appropriate stringency condition may be chosen for the hybridization step. “Stringency” is used in reference to the conditions of temperature, ionic strength, and the presence of other compounds, under which nucleic acid hybridizations are conducted. At “high stringency” conditions, nucleic acid base pairing will occur only between nucleic acid fragments that have a high frequency of complementary base sequences. At “weak” or “low” stringency, nucleic acids that are not completely complementary to one another will hybridize to one another. Because SNP alleles differ by only a single nucleotide, methods for detecting SNP alleles described herein normally comprise hybridization of the sample to the microarray under high stringency conditions. “Hybridization” involves the annealing of a complementary sequence to the target nucleic acid. The ability of two polymers of nucleic acid containing complementary sequences to find each other and anneal through base pairing interaction is a well-recognized phenomenon, and conditions for hybridization may be chosen and refined by a person skilled in the art. In some embodiments, the time for hybridization is about 30 minutes to about 48 hours, such as about any one of 30 minutes to 1 hour, 1 hour to 2 hours, 2 hours to 12 hours, 12 hours to 24 hours, or 18 hours to 24 hours.

The probe-target hybrids formed between the oligonucleotide probes and the target nucleic acids may be detected directly or indirectly. In some embodiments, the target nucleic acids are attached to a label that can be detected by any methods known in the art. In extension-based SNP detection methods, the oligonucleotide probe at the free terminus is extended by one or more nucleotides the terminal nucleotide in the oligonucleotide probe base pairs perfectly with the corresponding nucleotide in the target, indicating that a particular allele is present. Perfect complementarity between the rest of the oligonucleotide probe and the target nucleic acid enhances the rate of extension. Thus, labelled nucleotides, such as fluorescently labelled nucleotides or nucleotides attached to a hapten may be used to allow direct or indirect detection of an extended oligonucleotide probe, thereby allowing detection of the SNP allele in the target nucleic acid. In ligation-based SNP detection methods, the oligonucleotide probe on the microarray is ligated to a free adapter oligonucleotide when the target nucleic acid is perfectly complementary to the oligonucleotide probe and the free adapter oligonucleotide. Thus, the free adapter oligonucleotide may be labelled to allow detection of a ligated oligonucleotide probe on the microarray, thereby allowing detection of the SNP allele in the target nucleic acid. Alternatively, a fluorescent dye that specifically binds to single-stranded nucleic acids may be used to stain modified oligonucleotide probes after separating the target nucleic acids from the oligonucleotide probes. As oligonucleotide probes having extension or ligation products are longer than unmodified oligonucleotide probes, the modified oligonucleotide probes show a stronger signal than unmodified oligonucleotide probes, thereby allowing detection of SNPs in the target nucleic acid. In some embodiments, a fluorescent dye that specifically binds to double-stranded nucleic acids may be used to stain the modified probe-target hybrids. As extended probe-target hybrids have longer fragments of double-stranded nucleic acids than non-extended probe-target hybrids, signals from extended probe-target hybrids are stronger than unextended probe-target hybrids, thereby allowing detection of hybridized target nucleic acid or SNPs in the target nucleic acid. Dyes specific for single-stranded nucleic acids or double-stranded nucleic acids are known in the art, including, for example, SYBR® Gold, SYBR® Green, PICOGREEN®, OLIGREEN® and RIBOGREEN®.

The term “label” as used herein refers to any atom or molecule which can be used to provide a detectable (and preferably quantifiable) signal, and which can be attached to a nucleic acid. Labels may provide signals detectable by fluorescence, radioactivity, colorimetry, gravimetry, X-ray diffraction or absorption, magnetism, enzymatic activity, and the like. In some embodiments, the label is a hapten. “Hapten” refers to a small molecule, such as drug, hormone, or synthetic compound. A hapten may be detected by staining with a labelled protein, such as an antibody, that specifically recognizes the hapten. Non-limiting examples of label moieties useful for detection in the present application include, without limitation, suitable enzymes such as horseradish peroxidase, alkaline phosphatase, β-galactosidase, or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthamide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc⁹⁹, ³⁵S or ³H. For high-density microarrays, fluorescent labels can be conveniently used for detection, as fluorescent signals from a large number of test areas on the microarray may be detected simultaneously using a fluorescence microscope or a fluorescence scanner.

The primer extension assays, such as SBE and ASPE, and ligation assays used to detect SNPs in the target nucleic acids have been described in the art. See, for example, Gunderson K L et al. Nature Genetics, 2005, 37:S5; US20080131894A1; US20020177141A1; and US20030016897A1, which are incorporated herein by reference. Other SNP detection methods may alternatively be used with the microarrays described herein, including, but not limited to, rolling circle-based detection methods, allele-specific oligonucleotide (ASO) hybridization and others.

As the oligonucleotide probes on the SNP microarrays described herein in some embodiments are resistant to exonuclease, the extension-based SNP methods described herein may use a polymerase with proofreading activity, or the 3′ to 5′ exonuclease activity, which removes a wrongly incorporated nucleotide during the primer extension step. In some embodiments, the polymerase is a high fidelity DNA polymerase, such as Klenow fragment, T7, Pfu, Pow, Vent, or Pab phi29 DNA polymerase. By contrast, current SNP microarrays with normal DNA oligonucleotide probes are not compatible with primer extension assays using DNA polymerases having proofreading activities. Use of polymerases with proofreading activity can reduce noise from non-allele specific extension, thereby further improve accuracy of SNP detection in the methods described herein.

The methods described herein may include steps of regenerating the oligonucleotide probes or the microarrays after use, and/or reusing the oligonucleotide probes or the microarrays. Used SNP microarrays of the present application may be regenerated by subjecting the microarrays to the cleavage method, such as a chemical cleavage method or an exonuclease, which degrades the extended or ligated portion of the oligonucleotide probes on the microarray. Any damaged oligonucleotide probes that are missing the terminal nucleotides at the free terminus may also be degraded by the cleavage method, if the terminal nucleotide at the free terminus is the only cleavage-resistant nucleotide in each oligonucleotide probe. In some embodiments, the regeneration step comprises contacting the microarray with a 3′ to 5′ exonuclease, such as Exo III, Exo T, RecBCD (Exo V), Exo VI, BAL-31. The microarray may be treated with the cleavage method for at least any of 30 minutes, 1 hour, 2 hours, 3 hours, 6 hours, or 12 hours to completely remove all non-probe portions of the nucleic acids on the microarray and to remove all damaged oligonucleotide probes. In some embodiments, the hybridized target nucleic acid is removed from a used microarray by subjecting the microarray to a denaturing condition. Any denaturing agent or condition for separating two nucleic acid strands in the art may be used, and mild agents may be preferred to reduce damage to the oligonucleotide probes on the microarray. For example, the microarray may be washed with formamide and optionally heated to remove the hybridized target nucleic acid. In some embodiments, the microarray is heated to at least about any of 60° C., 65° C., 70° C., 75° C., 80° C., 85° C., 90° C. or more to denature any probe-target hybrids. In some embodiments, the regeneration step comprises first subjecting the microarray to a denaturing condition (such as formamide and/or heat to 85° C.), and then treating the microarray with an exonuclease (such as a 3′ to 5′ exonuclease). The regeneration step may further comprise washing steps to remove the denaturing agent and/or the exonuclease.

The microarrays of the present application may be used and regenerated for any number of times until accuracy of detection decreased by an observable level, for example, no more than about any of 20%, 15%, 10%, 8%, 6%, 5%, 4%, 3%, 2%, 1%, or less. The quality of signal may be determined using a reference sample having a known SNP allele that can be detected by a reference oligonucleotide probe on the SNP microarray. In some embodiments, the method comprises regenerating and reusing the microarray for at least about any of 2, 3, 4, 5, 10, 15, 20, 30, 40, 60, 70, 80, 90, 100, 200, 500, 1000 or more times. In some embodiments, the method comprises regenerating and reusing the microarray for about any one of 2-10, 10-20, 20-50, 50-100, 2-100, 100-500 or 500-1000 times.

As an example, a DNA sample (“target DNA”) can be hybridized to the exemplary SNP microarray comprising 24mer probes described in the section “SNP microarrays,” and an allele specific extension reaction is carried out using a proofreading DNA polymerase (T7, Pfu, etc.). The oligonucleotide probes on the microarray only extends if the 3′ terminal nucleotide is correctly base paired with the SNP nucleotide in the target DNA. The extension reaction is carried out with fluorescently labelled deoxyribonucleotides so that the extended probe is fluorescent. Because this is a one-color detection system, which uses 2 different probes to genotype each SNP, amplification of the signal can be achieved by means of incorporating many fluorescently labelled nucleotides into the strand, and the target nucleic acid can be >100 bp in length to enhance the fluorescent signal. For SNP detection purposes, one may only be concerned with whether the probe has extended or not, and not on determining what nucleotides have been incorporated. Compared to other methods of detection that uses a hapten-labelled nucleotide, use of fluorescently labelled nucleotides for detection of extended SNP probes avoid the need to use fluorescently labelled antibodies and several staining reactions, and thus may be both significantly faster and cheaper. Hapten modified nucleotides and labelled antibodies can be useful in methods that require further amplification of signal. Because of the phosphorothioate modification introduced into the last linkage in the oligonucleotide probe, a proofreading polymerase can be used in the primer extension reaction. Without this modification the 3′→5′ proofreading (exonuclease) activity of the polymerase can rapidly degrade the unprotected 3′ end of normal SNP DNA probes, halting the assay. By contrast, oligonucleotide probes having a phosphorothioate linkage between the 3′ terminal and penultimate nucleotides are stable for extended periods of time with common proofreading polymerases such as T7 and Pfu. Consequently, extension-based SNP detection method combining the microarray described herein and a polymerase with proofreading activity can greatly increase in the accuracy of SNP genotyping because the proofreading polymerase has a significantly lower rate of extension from mismatched base pairs than non-proofreading polymerases currently used in SNP genotyping assays. The polymerase in this case can serve as an OFF/ON switch and allows for high accuracy genotyping. See, for example, Di Gusto D. Nucleic Acids Research (2003) 31(3): e7; and Yang H L et al. Biochemical and Biophysical Research Communications (2005) 328: 265-272.

After use of the exemplary microarray for extension-based SNP detection, the target DNA can be stripped from the microarray with denaturing conditions such as high temperature (85° C.) in formamide and SDS with agitation, which removes hybridized target DNA from the oligonucleotide probes. Any oligonucleotide probes that are covalently modified due to the polymerase extension still remain modified. In order to regenerate the modified oligonucleotide probes, the microarray can be subsequently treated with a 3′→5′ Exonuclease (Exo I, Exo III or the like), which removes nucleotides from the 3′ end of the oligonucleotide probes until it reaches the phosphorothioate modified linkage that is stable to nuclease degradation. Thereby, the nucleotides incorporated by the polymerase in the extension reaction are removed and the oligonucleotide probes are reset to their initial states. After a further wash step to remove the exonuclease and nucleotide monomers that were removed, the microarray is completely regenerated and can be used again to detect SNPs in another sample.

### Kits and Articles of Manufacture

The present application further provides kits and articles of manufacture for using and reusing the oligonucleotide probes and microarrays described herein, useful for a variety of applications, including, but not limited to SNP detection using a primer extension assay or a ligation assay.

In some embodiments, there is provided a kit for detecting the presence or absence of a SNP allele in one or more samples of target nucleic acids, comprising: (a) an SNP microarray comprising: a plurality of single-stranded oligonucleotide probe pairs each comprising a first probe and a second probe, wherein each probe has a first terminus and a second terminus, wherein each probe is attached to a solid substrate via the first terminus, wherein the terminal nucleotide at the second terminus of each probe is resistant to cleavage, wherein the first probe and the second probe each comprises a matching (e.g., perfectly matching) sequence immediately upstream or immediately downstream of a single-nucleotide polymorphism (SNP), and wherein the terminal nucleotide at the second terminus of the first probe matches a first allele of the SNP and the terminal nucleotide at the second terminus of the second probe matches a second allele of the SNP; and (b) instructions for using and/or reusing the microarray. In some embodiments, the last two or more (e.g., the last 3, 4, 5 or more) nucleotides at the second terminus in each probe are resistant to cleavage. In some embodiments, the SNP microarray comprises at least about 200 (such as at least about any of 400, 600, 800, 1000, 10⁴, 10⁵, 10⁶, or more) oligonucleotide probe pairs each specific for a different SNP locus of interest. In some embodiments, the SNP microarray is used in combination with an extension assay, such as an allele-specific primer extension or single base extension assay, for SNP detection. In some embodiments, the SNP microarray is used in combination with an allele-specific ligation assay for SNP detection. In some embodiments, each oligonucleotide probe is about 20 to about 100 nucleotides long. In some embodiments, the plurality of oligonucleotide probes is attached to the same solid substrate (such as glass slide). In some embodiments, each oligonucleotide probe is attached to the solid substrate at a pre-determined position. In some embodiments, the plurality of oligonucleotide probes is attached to different solid substrates (such as beads). In some embodiments, each oligonucleotide probe pair is attached to the same solid substrate (such as bead).

In some embodiments, there is provided a kit for detecting the presence or absence of a SNP allele in one or more samples of target nucleic acids, comprising: (a) an extension-based SNP microarray comprising: a plurality of single-stranded oligonucleotide probes, wherein each oligonucleotide probe is attached to a solid substrate via the 5′ terminus, wherein the 3′ terminal nucleotide is linked to the penultimate nucleotide via a phosphorothioate group in the Sp configuration in each oligonucleotide probe, wherein the 3′ terminal nucleotide is the only nucleotide in each oligonucleotide probe that is resistant to a 3′ to 5′ exonuclease, and wherein at least two of the oligonucleotide probes on the microarray are different; and (b) instructions for using and/or reusing the microarray. In some embodiments, the last two or more (e.g., the last 3, 4, 5 or more) nucleotides at the 3′ terminus in each probe are each linked to their neighboring nucleotides via phosphorothioate groups in the Sp configuration. In some embodiments, the kit further comprises the 3′ to 5′ exonuclease, such as Exo III, Exo T, RecBCD (Exo V), Exo VI, BAL-31. In some embodiments, the kit further comprises a polymerase with proofreading activity, such as Klenow fragment, T7, Pfu, Pow, Vent, or Pab phi29 DNA polymerase. In some embodiments, the kit comprises fluorescently labelled (such as Cy3 labelled) nucleotides. In some embodiments, the kit comprises chain-terminating nucleotides comprising a hapten. In some embodiments, the kit comprises a fluorescently labelled protein (e.g., antibody) that specifically binds to the hapten. In some embodiments, at least about 200 (such as at least about any of 400, 600, 800, 1000, 10⁴, 10⁵, 10⁶, or more) oligonucleotide probes on the microarray are different. In some embodiments, each oligonucleotide probe is about 20 to about 100 nucleotides long. In some embodiments, the plurality of oligonucleotide probes is attached to the same solid substrate (such as glass slide). In some embodiments, the plurality of oligonucleotide probes is attached to different solid substrates (such as beads).

The kits may contain one or more additional components, such as containers, buffers, reagents, cofactors, or additional agents, such as agents for isolating nucleic acids from cells. The kits may also contain data analysis software or instructions for data analysis. The kit components may be packaged together and the package may contain or be accompanied by instructions for using the kit.

It will be appreciated by persons skilled in the art the numerous variations, combinations and/or modifications may be made to the invention as shown without departing from the spirit of the inventions as broadly described.

### EXAMPLES

The examples below are intended to be purely exemplary of the invention and should therefore not be considered to limit the invention in any way. The following examples and detailed description are offered by way of illustration and not by way of limitation.

### Example 1: Exonuclease Treatment to Reuse Probes Attached to a Glass Surface

This example demonstrates effective single nucleotide polymorphism (SNP) detection using oligonucleotide probes having a phosphorothioate linkage between the 3′ terminal and penultimate nucleotides, as well as regeneration of the probes by exonuclease treatment.

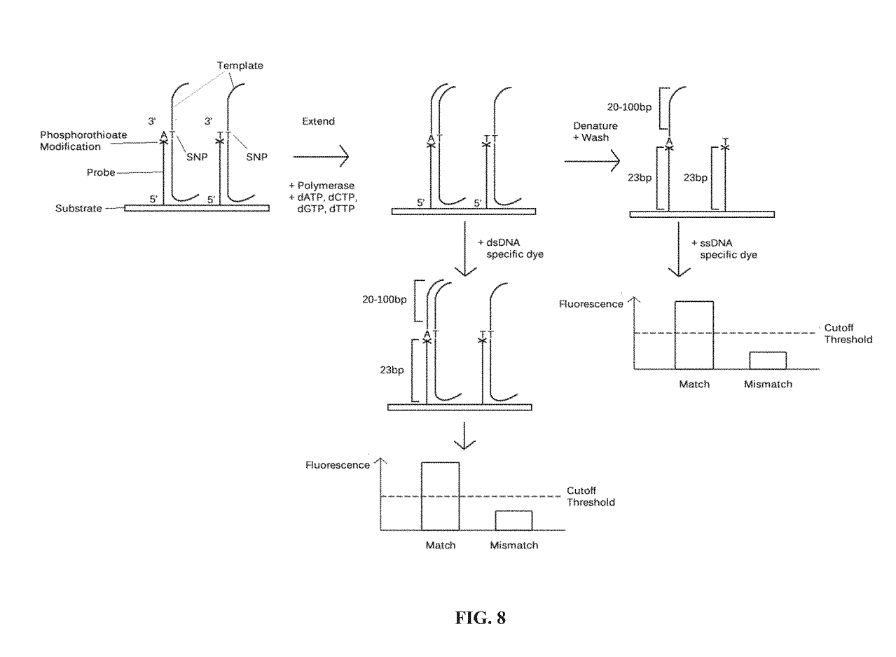

Three oligonucleotide probes were purchased from Integrated DNA Technologies (Coralville, Iowa, IDT) and are shown in Table 1. Each contained IDT's proprietary hydrazide modification at the 5′ end entitled the I-Linker and is represented by “/5ILink12/” in the table. “Probe A” and “Probe A*” are identical except Probe A* has a phosphorothioate linkage between the last two bases at the 3′ end, which is indicated by an asterisk between the two bases in the table. In contrast, Probe A has a natural phosphodiester linkage. Probe A* and Probe B* differ only in the terminal base at the 3′ end. These probes were attached to epoxy coated silanized glass slides provided by AutoMate Scientific, Inc. (Berkeley, Calif.). The probes were each diluted to 10 μM concentration in 100 mM sodium phosphate, monobasic buffer and spotted on the epoxy slides by touching the tip of a 20 μL pipette that had been loaded with 3 μL of the probe to the slide surface. The probes were spotted in the geometry shown in FIG. 10. 100 mM sodium phosphate, monobasic buffer was used for the “no probe control” spot. Mineral oil was immediately overlaid on each region of the three probes to prevent evaporation and the slide was incubated at 27° C. for one hour.

Slides with attached probes were then washed twice with a solution containing 1% Tween-20 and 2×SSC (1×SSC contains 150 mM sodium chloride, and 15 mM sodium citrate and is adjusted to a pH of 7.0 using hydrochloric acid), then washed again with 0.1% Tween-20 and 2×SSC, then 0.2×SSC, and finally 0.02×SSC. Slides were dried using a stream of compressed nitrogen.

A synthetic, double-stranded DNA template was prepared by annealing a longer oligonucleotide in which the central region is complementary to Probe A (“Template A”) and its complement (“Template Ac”, both purchased from IDT and listed in Table 1). The DNA templates were each to 200 μM in 1×TE buffer (10 mM Tris-HCl pH 8, 1 mM EDTA pH 8.0), mixed in equal volumes, placed for 30 minutes on a heat block that had been heated to 95° C., and the heat block was allowed to cool to room temperature over the course of several hours. This double-stranded template (“Template Ads”) was used to prepare 100 nM Template Ads in 5×SSC, heated at 95° C. for 6 minutes, immediately placed on ice for 2 minutes, then hybridized to the immobilized probes on the washed, dried slides by overlaying them on top of the region where the probes had been attached, followed by incubation in a hybridization chamber (a hermetically sealed plastic chamber containing wet tissue to provide adequate humidity) at 42° C. for 120 minutes. The slides were then washed in 2×SSC, 0.2×SSC, and 0.02×SSC and dried again under a stream of nitrogen.

TABLE 1
Oligonucleotide probes.

| Oligonucleotide | Sequence (5′-3′) |
|---|---|
| Probe A | /5ILINK12/CTCGGCAAAATTTCACCTATTA |
| | CTGTCATCAGTCAGTGGCCTGGTATGATA |
| | (SEQ ID NO: 1) |
| Probe A* | /5ILINK12/CTCGGCAAAATTTCACCTATTA |
| | CTGTCATCAGTCAGTGGCCTGGTATGAT*A |
| | (SEQ ID NO: 2) |
| Probe B | /5ILINK12/CTCGGCAAAATTTCACCTATTA |
| | CTGTCATCAGTCAGTGGCCTGGTATGATG |
| | (SEQ ID NO: 3) |
| Probe B* | /5ILINK12/CTCGGCAAAATTTCACCTATTA |
| | CTGTCATCAGTCAGTGGCCTGGTATGAT*G |
| | (SEQ ID NO: 4) |
| Template A | CCCAAGAACCGAAGTGCTTTGTCATAGCTTCC |
| | TGATGACATATCATACCAGGCCACTGACTGAT |
| | GACAGTAATAGGTGAAATTTTGCCGA |
| | (SEQ ID NO: 5) |
| Template Ac | TCGGCAAAATTTCACCTATTACTGTCATCAGT |
| | CAGTGGCCTGGTATGATATGTCATCAGGAAGC |
| | TATGACAAAGCACTTCGGTTCTTGGGG |
| | (SEQ ID NO: 6) |
| Template B | ATCATTTGATCCCAAGAACCGAAGTGCTTTGT |
| | CATAGCTTCCTGATGACACATCATACCAGGCC |
| | ACTGACTGATGACAGTAATAGGTGAAATTTTG |
| | CCGA |
| | (SEQ ID NO: 7) |
| Template Bc | TCGGCAAAATTTCACCTATTACTGTCATCAGT |
| | CAGTGGCCTGGTATGATGTGTCATCAGGAAGC |
| | TATGACAAAGCACTTCGGTTCTTGGGATCAAA |
| | TGAT |
| | (SEQ ID NO: 8) |

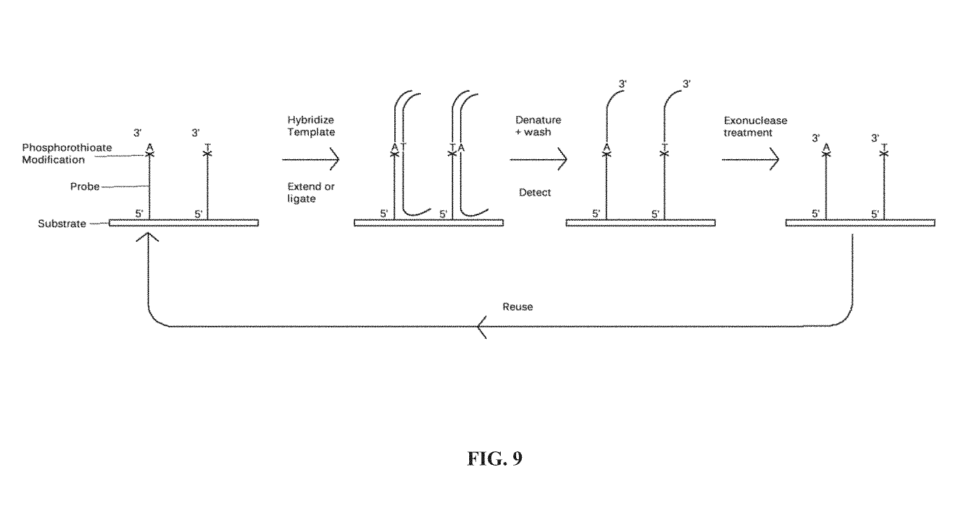

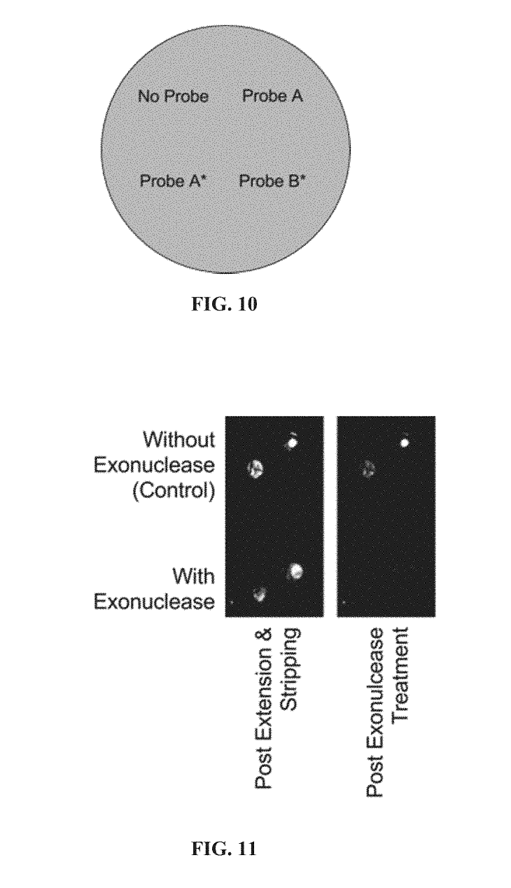

The immobilized probes on washed, dried slides with hybridized DNA were then extended by overlaying a solution of 1× Taq PCR buffer (MCLAB, South San Francisco, Calif.), 50 μM dCTP, 50 μM dGTP, 50 μM dATP, 10 μM 5-(3-Aminoallyl)-2′-deoxyuridine-5′-triphosphate, labeled with Cy3 (Jena Biosciences, Jena, Germany), 0.04 U/μL Taq-Klenow DNA Polymerase (Molecular Cloning Laboratories), and 8.2 μg/mL single-stranded binding protein (MCLAB), followed by incubation of the slide for 7 minutes at 42° C. The slide was then washed and dried as in the hybridization step, washed again in 0.1 N sodium hydroxide to remove the hybridized template, washed three times in distilled water, dried, and imaged on microarray scanner (PROSCANARRAY™, PerkinElmer, Waltham, Mass.) using the Cy3 channel and a 70% PMT setting. The image of the slide is shown in left panel of FIG. 11.

The spots were then overlaid with a solution containing 4 U/μL Exonuclease I (NEB, Ipswich, Mass.), in 67 mM Glycine, 6.7 mM magnesium chloride, 10 mM beta-mercaptoethanol, 2.5ng/μl bovine serum albumin (BSA) that had the pH adjusted to 9.5 using potassium hydroxide and incubated for 210 minutes at 37° C. After the same washing and drying procedure as in the extension step, the slide was imaged again. The image of the slide is shown in the bottom row of the right panel in FIG. 11. A control reaction, which omits only the exonuclease in the treatment is shown in the top row of the right panel in FIG. 11.

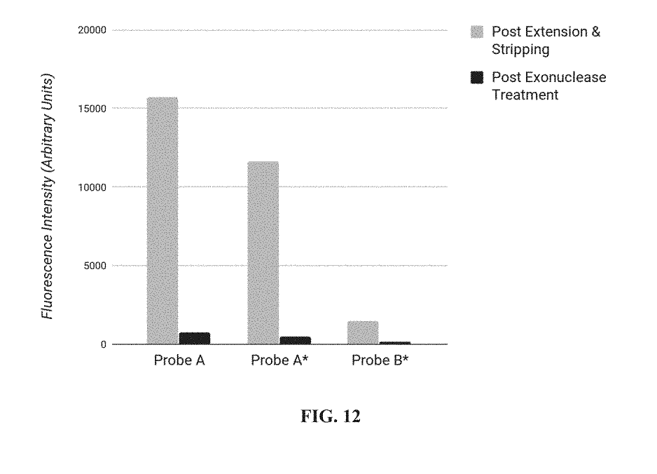

Image analysis was performed by using open-source software (ImageJ). The average fluorescence intensity of each spot was quantified. The intensity directly after extension and stripping, and after exonuclease treatment is graphed in FIG. 12.

The results of FIGS. 11 and 12 demonstrate: (1) Extension reactions generally perform better off of non-phosphorothioate probes than those with a phosphorothioate linkage between the 3′ terminal and penultimate base; (2) The above hybridization and extension conditions allow for the discrimination of a single base difference if that difference is at the 3′ end of the probe; and (3) The above exonuclease treatment conditions degraded the extension products by more than tenfold.

### Example 2: Repeated Single Nucleotide Polymorphism (SNP) Discrimination in Synthetic Templates by Reusable Oligonucleotide Probes

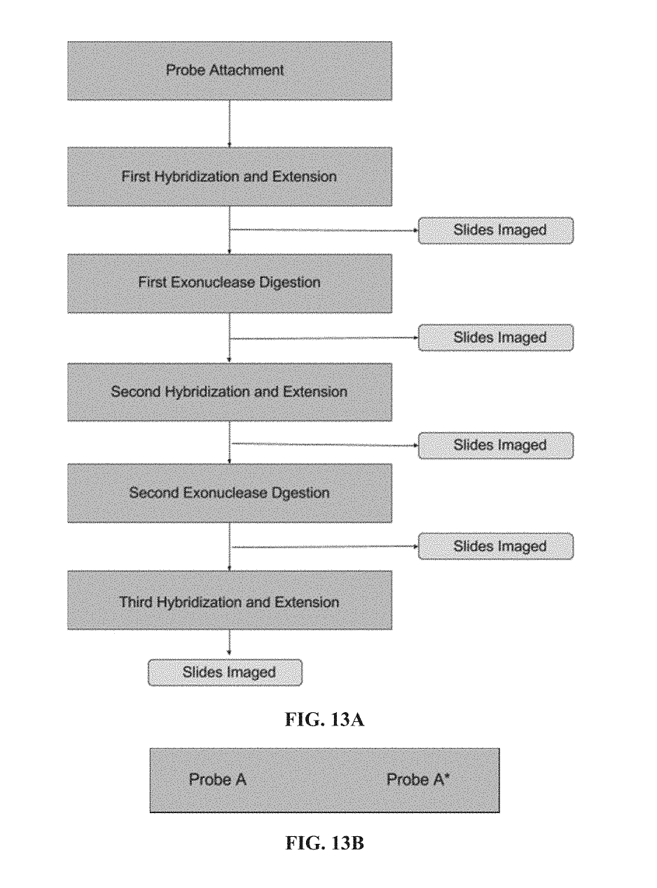

This example demonstrates reuse of oligonucleotide probes for SNP detection. FIG. 13A illustrates the workflow of the experimental procedure in this Example.

### A. Experiment 1

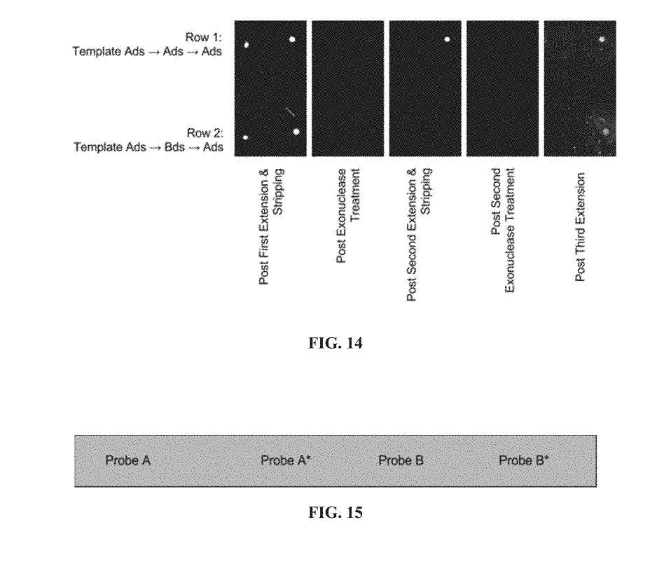

Hydrazide-modified oligonucleotide probes were attached to an epoxy-coated glass slide by the same procedure as described in Example 1. Probes were attached in a straight row as shown in FIG. 13B. See Table 1 for the probe sequences. Hybridization and extension on slide was done using 10 nM of Template Ads in 5×SSC for hybridization using the same procedure as described in Example 1. Slide was imaged on microarray scanner using the Cy3 channel and a 60% PMT setting. The image of the slide is shown in the first panel from on left in FIG. 14.

The spots were then overlaid with a solution containing 2 U/μL Exonuclease T (NEB), in 50 mM potassium acetate, 20 mM Tris-acetate, 10 mM magnesium acetate, 1 mM DTT, 2.5 ng/μl BSA that had the pH adjusted to 7.9 and incubated for 120 minutes at 25° C. After the same washing and drying (i.e., stripping) procedure as described in Example 1, the slide was imaged again using the Cy3 channel and a 60% PMT setting. The image of the slide is shown in the second panel from the left in FIG. 14.

A synthetic, double-stranded DNA template (Template Bds) was prepared by annealing a longer oligonucleotide in which the central region is complementary to Probe B (“Template B”) and its complement (“Template Bc”, both purchased from IDT and listed in Table 1), using the same procedure as described in Example 1. 100 nM Template Ads or 100 nM of Template Bds in 5×SSC was used for a second round of hybridization to attached probes in Row 1 and Row 2 respectively, using the same procedure as described in Example 1. A second extension was performed by the same procedure as described in Example 1 for 10 minutes at 42° C. The slide was once again washed in 2×SSC, 0.2×SSC, and 0.02×SSC, dried, and imaged. The image of the slide is shown in the third panel from the left in FIG. 14.

A second exonuclease treatment was carried out on the slide using Exonuclease T by the same procedure as the first exonuclease treatment, except the incubation was for 110 min at 25° C. The slide was then imaged on the microarray scanner using the Cy3 channel and a 60% PMT setting. The image of the slide is shown in the fourth panel from the left in FIG. 14.

100 nM of Template Ads in 5×SSC was used for a third hybridization on both Row 1 and Row 2 by the same procedure as described in Example 1. A third extension was then performed by once again overlaying a solution of 1× Taq PCR buffer (MCLAB), 50 μM dCTP, 50 μM dGTP, 50 μM dATP, 10 μM 5-(3-Aminoallyl)-2′-deoxyuridine-5′-triphosphate, labeled with Cy3 (Jena Biosciences), 0.04 U/μL Taq-Klenow DNA Polymerase (MCLAB), and 8.2 μg/mL single-stranded binding protein (MCLAB) and incubating the slide for 10 minutes at 42° C. The slide was imaged on the microarray scanner using the Cy3 channel and a 60% PMT setting. The image of the slide is shown in the fifth panel from the left in FIG. 14.

The brightness and contrast were adjusted individually for each image in FIG. 14. The following algorithm was applied to the data to call out SNPs via an automatic process: (1) The background of each image (column) was determined by measuring the intensity in a region surrounding the region of amplification; (2) The background value was subtracted from the value from the region of interest; (3) Numbers lower than background were reported as “<0”. Results are shown in Table 2 below.

TABLE 2
Background corrected intensity ratios of reusable probes in experiment 1.

| | Probe A | Probe A* | Probe A | Probe A* |
|---|---|---|---|---|
| | (Row 1) | (Row 1) | (Row 2) | (Row 2) |
| Post Extension and Stripping | 10186 | 4769 | 11642 | 3422 |
| Post Exonuclease Treatment | 28 | 476 | 104 | 402 |
| Post Second Extension and | <0 | 5023 | <0 | 132 |
| Stripping | | | | |
| Post Second Exonuclease | <0 | 143 | <0 | 16 |
| Treatment | | | | |
| Post Third Extension | <0 | 580 | <0 | 371 |

### B. Experiment 2

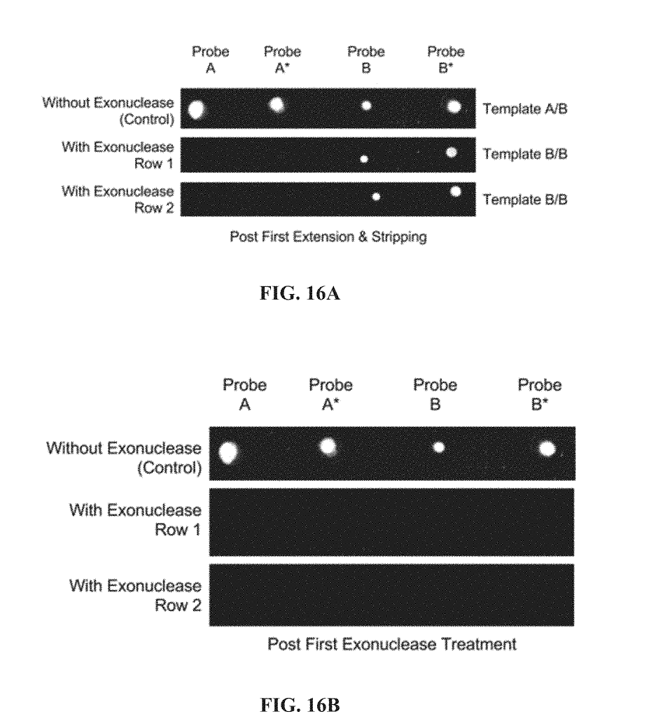

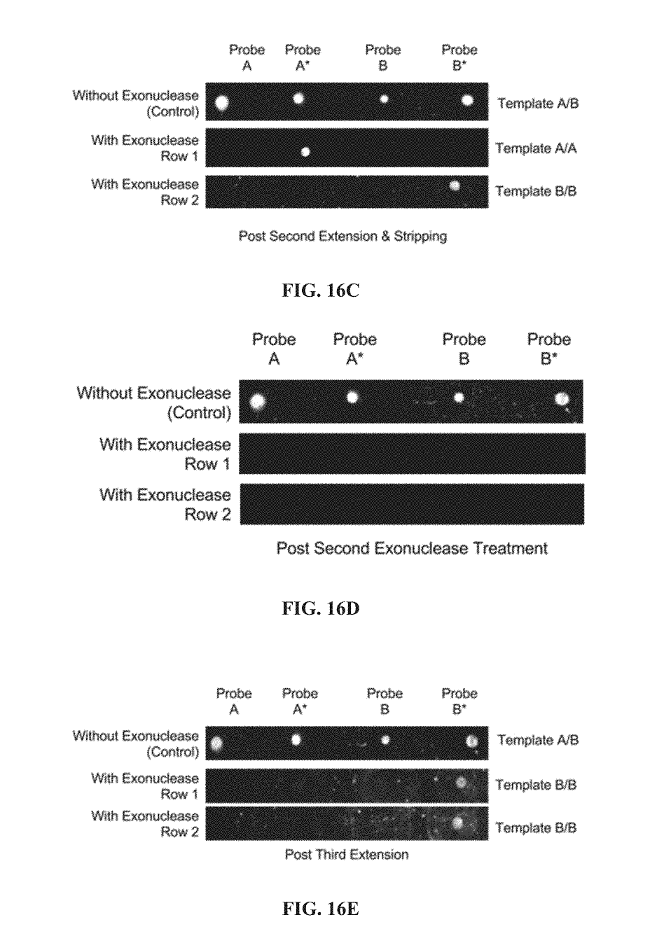

Hydrazide-modified oligonucleotide probes were attached to an epoxy-coated slide by the same procedure as described in Example 1. Probes were attached in a straight row as shown in FIG. 15. See Table 1 for the probe sequences. Repeated hybridization, extension and exonuclease reactions were carried out on the slide by the same procedure as described in Section A of this example. For the first hybridization, 10 nM of Template Bds in 5×SSC was used for both Rows 1 and 2. For the second hybridization, 100 nM Template Ads or 100 nM of Template Bds in 5×SSC was used on Row 1 or Row 2 respectively. 100 nM of Template Bds in 5×SSC was used for the third hybridization on both rows 1 and 2. FIGS. 16A-16E show images of the slide taken after each extension and exonuclease reaction.

The top rows in FIG. 16A-16E show control reactions in which the exonuclease was omitted in the exonuclease treatment step. The bottom rows were processed as described above. The background corrected ratios comparing the extension products with the prior, exonuclease-treated ones (as discussed in section A), are shown in Table 3, whereas those for the controls are shown in Table 4 below.

TABLE 3
Background corrected intensities of reusable probes in Experiment 2.

| | Row | | | | | | | |
|---|---|---|---|---|---|---|---|---|
| | Row 1 | | Row 2 | | Row 1 | | Row 2 | |
| Probe | A | A* | A | A* | B | B* | B | B* |
| Post Extension and | <0 | 53 | <0 | 45 | 4442 | 2130 | 5532 | 3048 |
| Stripping | | | | | | | | |
| Post Exonuclease | <0 | 43 | <0 | 45 | 469 | 352 | 473 | 539 |
| Treatment | | | | | | | | |
| Post Second Extension | <0 | 47574 | <0 | <0 | 43 | 196 | 9 | 387 |
| and Stripping | | | | | | | | |
| Post Second Exonuclease | <0 | <0 | <0 | <0 | <0 | <0 | 95 | 60 |
| Treatment | | | | | | | | |
| Post Third Extension | <0 | <0 | <0 | <0 | <0 | 232 | 64 | 316 |

TABLE 4
Background corrected intensities in control of Experiment 2.

| | Probe A | Probe A* | Probe B | Probe B* |
|---|---|---|---|---|
| | (Control) | (Control) | (Control) | (Control) |
| Post Extension and | 6561 | 7654 | 5071 | 4535 |
| Stripping | | | | |
| Post Exonuclease | 4848 | 5481 | 3460 | 3074 |
| Treatment | | | | |
| Post Second Extension | 1563 | 1935 | 1408 | 2516 |
| and Stripping | | | | |
| Post Second Exonuclease | 428 | 700 | 687 | 818 |
| Treatment | | | | |
| Post Third Extension | 327 | 635 | 550 | 486 |

### C. Experiment 3

Hydrazide-modified oligonucleotide probes were attached to an epoxy-coated slide by the same procedure as described in Example 1. Probes were attached in a straight row as shown in FIG. 15. Repeated hybridization, extension and exonuclease reactions were carried out on the slide by the same procedure as described in section A of this example. For the first hybridization, a mixture of 10 nM of Template Ads and 10 nM of Template Bds in 5×SSC were used. For the second hybridization 100 nM Template Ads or 100 nM of Template Bds in 5×SSC was used on Row 1 or Row 2 respectively. A mixture of 100 nM of Template Ads and 100 nM of Template Bds in 5×SSC was used for the third hybridization on both Rows 1 and 2.

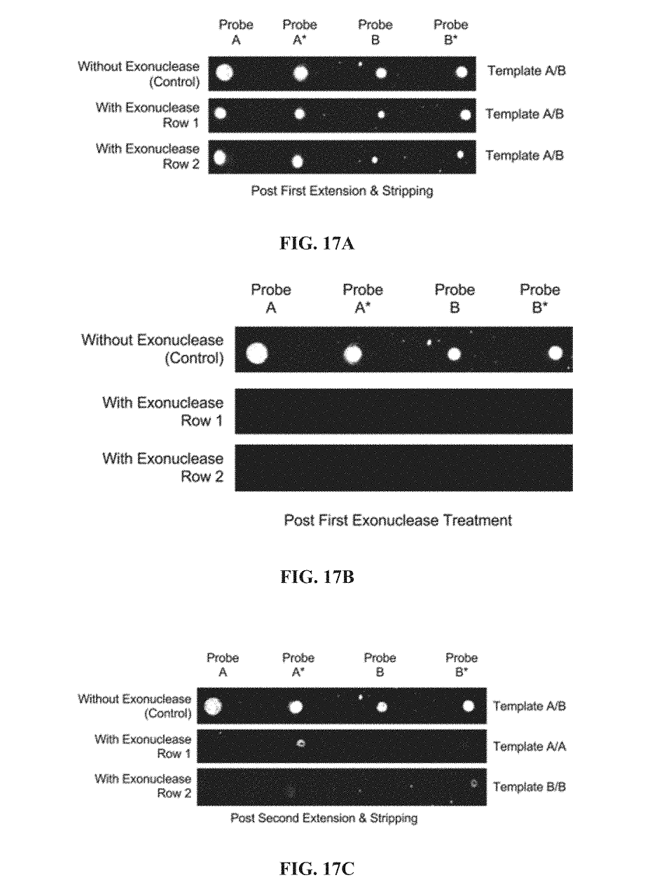

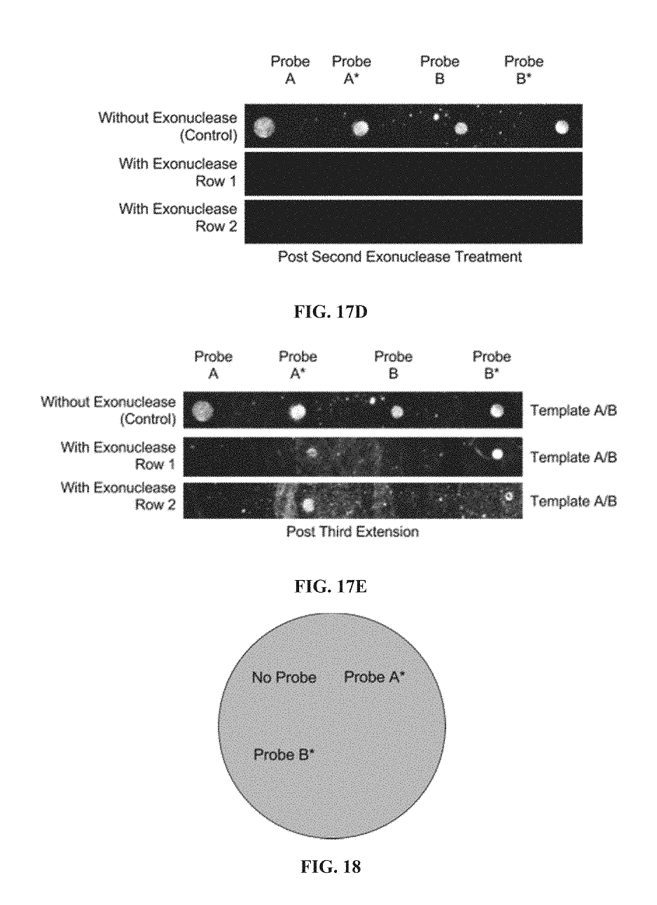

FIG. 17A-17E show images of the slides taken after each extension and exonuclease reaction. The top row of FIGS. 17A-17E show control reactions in which exonuclease was omitted in the exonuclease step. The bottom rows were processed as described above. The background corrected ratios comparing the extension products with the prior, exonuclease-treated ones (as discussed in section A), are shown in Table 5, whereas those for the controls are shown in Table 6 below.

TABLE 5
Background corrected intensities of reusable probes in Experiment 3.

| | Row | | | | | | | |
|---|---|---|---|---|---|---|---|---|
| | Row 1 | | | | Row 2 | | | |
| Probe | A | A* | B | B* | A | A* | B | B* |
| Post Extension and | 6927 | 5428 | 4499 | 6614 | 13132 | 3080 | 2359 | 4855 |
| Stripping | | | | | | | | |
| Post Exonuclease | <0 | 348 | 314 | 583 | 34 | 217 | 109 | 139 |
| Treatment | | | | | | | | |
| Post Second Extension | 1 | 2171 | 3 | 236 | 21 | 135 | <0 | 464 |
| and Stripping | | | | | | | | |
| Post Second Exonuclease | <0 | 96 | 51 | 157 | <0 | 87 | <0 | 163 |
| Treatment | | | | | | | | |
| Post Third Extension | <0 | 294 | 7 | 811 | <0 | 242 | <0 | 315 |

TABLE 6
Background corrected intensities in control of Experiment 3.

| | Probe A | Probe A* | Probe B | Probe B* |
|---|---|---|---|---|
| | (Control) | (Control) | (Control) | (Control) |
| Post Extension and | 1380 | 2693 | 3695 | 3533 |
| Stripping | | | | |
| Post Exonuclease | 1435 | 2227 | 2308 | 2396 |
| Treatment | | | | |
| Post Second Extension | 647 | 922 | 660 | 1328 |
| and Stripping | | | | |
| Post Second Exonuclease | 244 | 317 | 336 | 444 |
| Treatment | | | | |
| Post Third Extension | 114 | 281 | 207 | 232 |

The results of the experiments 1-3 demonstrate the following: (1) In the first extension, signals were only observed from Probe B or Probe B*, not Probe A or Probe A* (FIG. 17A). Consistent with the observations in Example 1, this result demonstrates that the conditions used in this experiment allowed for SNP discrimination. (2) Signals from Probe A or Probe B did not return after exonuclease treatment followed by another hybridization with the same template, and an identical extension reaction, whereas the signals did return with Probe A* and Probe B*, which contain a phosphorothioate linkage between the terminal two bases at the 3′ ends. This indicates that the phosphorothioate bond protects the probes from exonuclease degradation, and allows the exonuclease-treated probes to participate in another hybridization and extension reaction. (3) If exonuclease digestion proceeded but stopped before reaching the 3′ end of the probe, signals would arise from both non-phosphorothioate-containing and phosphorothioate-containing probes. The results here indicate that the conditions described in this example lead to full exonuclease digestion of all nucleotides extending from the 3′ end of the probes in the first extension reaction. (4) If exonuclease digestion could partially degrade the phosphorothioate linkage, such degradation would degrade non-specific phosphorothioate probes as well. Since both Probe A* and Probe B* are identical except for the 3′ terminal base, if partial degradation of the phosphorothioate linkage occurs, a second extension would occur off both partially degraded Probe A* and Probe B* in each example, compromising specificity of the signals. The conditions described in this example do not result in appreciable degradation of phosphorothioate probes, as different SNPs were successfully discriminated using the same probes for multiple times. (5) Experiments 1-3 show that the reusable probes and SNP detection methods described herein could be used to determine allele contents, including mixed/heterozygous alleles, using the same slide, for multiple synthetic templates hybridized sequentially after exonuclease treatment. Specifically, Experiment 1 in section A demonstrates the ability to assay one allele from a particular locus using one probe from three templates sequentially. Experiment 2 in section B demonstrates the ability to measure two different alleles from a locus using two probes up to three sequential times. Experiment 3 in section C demonstrates the ability to distinguish all versions of a biallelic locus, including the heterozygote, using two probes up to three times.

### Example 3: Repeated SNP Determination in Bovine Genomic DNA by Reusable Probes

This example demonstrates that single nucleotide polymorphisms can be detected in a specific manner sequentially from multiple genomic DNA samples on a single slide with reusable oligonucleotide probes. No new probes needed to be added to the slide for repeated SNP detection.

Genomic DNA was isolated from a bovine ear punch collected in an Allflex TSU (Tissue Sampling Unit, Allflex, Irving Tex.) provided via a commercial dairy farmer. The DNA was extracted using the QIAAMP® DNA Mini Kit (Qiagen) according to the manufacturer's instructions and resuspended in TE buffer (10 mM Tris, 1 mM EDTA, pH adjusted to 8.0 with HCl). The extracted DNA from samples representing one homozygous and one heterozygous versions of the Bos taurus SNP rs 110424885 (shown in Table 7 below) were used for further analysis.

TABLE 7
SNP genotype.

| Sample Name | SNP ID | Genotype |
|---|---|---|
| H735 | rs110424885 | C/C |
| H178 | rs110424885 | C/T |

The extracted genomic DNA was amplified using the REPLI-G® maxi kit (Qiagen) in 1 mL volume according to the manufacturer's instructions. The amplified DNA was then fragmented using 500 μL DNA, 0.25 U DNase I (NEB), 0.528 mM calcium chloride, and 1× reaction buffer as provided by the manufacturer, in a total volume of 900 μL. The reaction was incubated at 37° C. for 15 minutes, deactivated by adding 100 μL of 0.1% SDS and incubation at 95° C. for 15 minutes, and then stored on ice. The resulting final concentration of amplified DNA was between 450 and 550 ng/μL.

The fragmentation reaction was cleaned up by performing a standard isopropanol precipitation. 3M sodium acetate (pH 5.2) was added to the fragmentation reaction in a volume equal to one-fifth the volume of the fragmentation reaction. Then isopropanol was added in equal volume to the reaction, mixed well, and incubated at −80° C. for three hours. After incubation, the sample was centrifuged at 16 g (maximum speed) for 35 minutes in an EPPENDORF® 5415R refrigerated centrifuge at 4° C. This produced a DNA pellet at the bottom of the tube. The supernatant was then carefully removed, the pellet washed with 1 mL ice-cold 70% ethanol, and centrifuged at the maximum speed at 4° C. for 10 minutes. This once again formed a pellet. The supernatant was once again removed and the pellet was allowed to dry. The pellet was then re-suspended in 100 μL hybridization buffer containing 1 M NaCl, 20% formamide, 100 mM potassium phosphate pH 7.5, 0.1% Tween-20 and incubated at 75° C. for 20 minutes to dissolve the pellet. The final concentration of DNA in the hybridization buffer was approximately 900 ng/μL.

Hydrazide-modified oligonucleotide probes were attached to an epoxy-coated glass slide by the same procedure as described in Example 1. Probes were attached according to the geometry shown in FIG. 18. See Table 1 for the probe sequences.

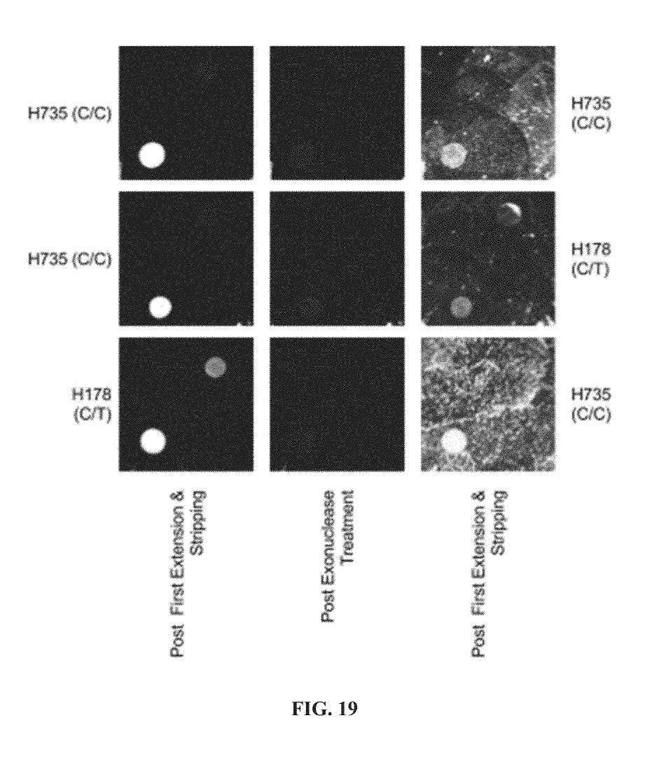

Fragmented, cleaned, and resuspended DNA was then pre-incubated at 95° C. for 20 minutes followed by incubation at 48° C. for 10 minutes, and placed on the slide, overlaying the probe spots. The slide was placed in a hybridization chamber (ArrayIt Corporation, Sunnyvale, Calif.), which contained 5 μL of water in the bottom of the chamber to saturate the air with water vapor, and had been pre-incubated at 48° C. The slide, in the chamber, with the hybridization solution overlaid on it, were then placed in a hybridization oven set to 48° C. for 22 hours. The specific genomic DNA placed on the slide is indicated on the left of the leftmost panels in FIG. 19. After hybridization the slide was washed with 2×SSC, 0.2×SSC, and 0.02×SSC and dried. Extension reaction was carried out on the slide by the same procedure as described in Example 1. The slide was then washed with 0.1 N sodium hydroxide to remove the hybridized template, washed with 2×SSC, 0.2×SSC, and 0.02×SSC, then dried. Images of the slides after the first extension and stripping are shown in the leftmost panels of FIG. 19.

Exonuclease treatment of the slide was performed as in Example 2 for 6 hours. The images of the slides after the exonuclease treatment are shown in the middle panels of FIG. 19.

A second round of hybridization and extension were then performed as described above using the templates indicated on the right of the rightmost panels in FIG. 19. Images of the slides after the second extension and stripping are shown in the rightmost panels of FIG. 19.

The raw intensity values, corrected for the background as described in section A of Example 2, are shown in Table 8 below.

TABLE 8
Background corrected intensity ratios of reusable probes.

| | Probe A* | Probe B* | Probe A* | Probe B* | Probe A* | Probe B* |
|---|---|---|---|---|---|---|
| | (Row 1) | (Row 1) | (Row 2) | (Row 2) | (Row 3) | (Row 3) |
| Second Extension: | <0 | 1.6 | <0 | 1.2 | <0 | 1.7 |
| Exonuclease treatment | | | | | | |

These results demonstrate efficient and repeated SNP detection in complex DNA samples using an array of reusable probes. In general, phosphorothioate linkage in the probes protects them during exonuclease treatment and allows for repeated genotyping using the same probes on the same slide. This example demonstrates that the reusable probes and SNP detection methods described herein are applicable to genomic DNA, which is much more complex than the synthetic DNA of Example 2. Differential signals from different rows on the slide in FIG. 19 indicate that the methods described herein can be used to genotype genomic DNA of varying compositions on the same slide, sequentially. Specifically, the top panels of FIG. 19 demonstrate the ability of using the probes to detect an organism homozygous at a particular locus, followed by one of the same genotype at that locus. The middle panels of FIG. 19 demonstrate the ability of using the probes to detect an organism homozygous at a particular locus, followed by one that is heterozygous at that locus. The bottom panels of FIG. 19 demonstrate the ability of using the probes to detect an organism heterozygous at a particular locus, followed by one that is homozygous at that locus.

What is claimed is:

1. A microarray comprising a plurality of single-stranded oligonucleotide probes each having a first terminus and a second terminus,
wherein each oligonucleotide probe is attached to a solid substrate via the first terminus, wherein the terminal nucleotide at the second terminus of each oligonucleotide probe is resistant to cleavage, and wherein at least two of the oligonucleotide probes are different.

2. The microarray of claim 1, wherein the terminal nucleotide at the second terminus of each oligonucleotide probe is resistant to a nuclease.

3. The microarray of claim 2, wherein the nuclease is an exonuclease.

4. The microarray of claim 1, wherein the first terminus of each oligonucleotide probe is the 5′ terminus.

5. The microarray of claim 1, wherein the microarray comprises at least about 200 different oligonucleotide probes.

6. The microarray of claim 1, wherein the terminal nucleotide at the second terminus of each oligonucleotide probe is a modified nucleotide having a modified linkage to the penultimate nucleotide.

7. The microarray of claim 6, wherein the terminal nucleotide at the second terminus of each oligonucleotide probe is linked to the penultimate nucleotide via a phosphorothioate group.

8. The microarray of claim 7, wherein the phosphorothioate group has Sp configuration.

9. The microarray of claim 1, wherein the terminal nucleotide at the second terminus is the only cleavage-resistant nucleotide in each oligonucleotide probe.

10. The microarray of claim 1, wherein the first two or more nucleotides at the second terminus in each oligonucleotide probe are resistant to cleavage.

11. The microarray of claim 10, wherein the first two or more nucleotides at the second terminus in each oligonucleotide probe are modified nucleotides having modified linkages to neighboring nucleotides.

12. The microarray of claim 11, wherein the first two or more nucleotides at the second terminus in each oligonucleotide probe are each linked to their neighboring nucleotides via phosphorothioate groups.

13. The microarray of claim 1, wherein each oligonucleotide probe is about 20 nucleotides to about 100 nucleotides long.

14. The microarray of claim 1, wherein each oligonucleotide probe is attached to the solid substrate at a pre-determined position on the solid substrate.

15. The microarray of claim 1, wherein each oligonucleotide probe is attached to the solid substrate at a random position on the solid substrate.

16. The microarray of claim 1, wherein the plurality of oligonucleotide probes comprises one or more oligonucleotide probe pairs, wherein each oligonucleotide probe pair comprises a first probe and a second probe each comprising a matching sequence immediately upstream or immediately downstream of a single-nucleotide polymorphism (SNP), and wherein the terminal nucleotide at the second terminus of the first probe matches a first allele of the SNP and the terminal nucleotide at the second terminus of the second probe matches a second allele of the SNP.

17. The microarray of claim 1, further comprising a plurality of target nucleic acids, wherein the target nucleic acids are hybridized to at least a portion of the oligonucleotide probes.

18. A method of preparing a reusable microarray, comprising: synthesizing a plurality of single-stranded oligonucleotide probes each having a first terminus and a second terminus on a solid substrate, wherein the first terminus of each oligonucleotide probe is attached to the solid substrate, and wherein the terminal nucleotide at the second terminus of each oligonucleotide probe is resistant to cleavage, and wherein at least two of the oligonucleotide probes are different, thereby providing the reusable microarray.

19. A method of detecting the presence or absence of a single-nucleotide polymorphic (SNP) allele in a target nucleic acid, comprising:
(a) hybridizing the target nucleic acid to the oligonucleotide probes of the microarray of claim 1 to provide probe-target hybrids, wherein the first terminus is the 5′ terminus, and wherein at least one oligonucleotide probe comprises a sequence that matches the SNP allele;
(b) contacting the probe-target hybrids with a polymerase and nucleotides under a condition that allows primer extension to provide modified probe-target hybrids; and
(c) detecting the modified probe-target hybrids thereby detecting the presence or absence of the SNP allele in the target nucleic acid.

20. The method of claim 19, wherein the polymerase is a polymerase with proofreading activity.

21. The method of claim 19, wherein the primer extension is allele-specific primer extension (ASPE).

22. The method of claim 21, wherein the nucleotides are fluorescently labelled, and wherein the modified probe-target hybrids are detected by detecting fluorescence from the nucleotides incorporated in the modified probe-target hybrids.

23. The method of claim 19, wherein the primer extension is single base extension (SBE).

24. A method of detecting the presence or absence of a single-nucleotide polymorphic (SNP) allele in a target nucleic acid, comprising:
(a) hybridizing the target nucleic acid to the oligonucleotide probes of the microarray of claim 1 to provide probe-target hybrids, wherein at least one oligonucleotide probe comprises a sequence that matches the SNP allele;
(b) contacting the probe-target hybrids with a ligase in the presence of a plurality of free adapter oligonucleotides under a condition that allows allele-specific ligation to provide modified probe-target hybrids; and
(c) detecting the modified probe-target hybrids thereby detecting the presence or absence of the SNP allele in the target nucleic acid.

25. The method of claim 19, further comprising (d) treating the microarray by contacting the microarray with an exonuclease.

26. The method of claim 25, wherein the treating further comprises subjecting the microarray to a denaturing condition.

27. The method of claim 19, further comprising (e) reusing the microarray according to steps (a)-(c).


SEQUENCE LISTING (8 sequences)

SEQ ID NO: 1 - DNA, 51 bp, Artificial Sequence
  Synthetic Construct
  misc_feature 1: Proprietary hydrazide modification at the 5′ end entitled the I-Linker
ctcggcaaaatttcacctattactgtcatcagtcagtggcctggtatgata

SEQ ID NO: 2 - DNA, 51 bp, Artificial Sequence; identical to SEQ ID NO: 1
  Synthetic Construct
  misc_feature 50,51: Joined by a phosphorothioate linkage
  misc_feature 1: Proprietary hydrazide modification at the 5′ end entitled the I-Linker

SEQ ID NO: 3 - DNA, 51 bp, Artificial Sequence
  Synthetic Construct
  misc_feature 1: Proprietary hydrazide modification at the 5′ end entitled the I-Linker
ctcggcaaaatttcacctattactgtcatcagtcagtggcctggtatgatg

SEQ ID NO: 4 - DNA, 51 bp, Artificial Sequence; identical to SEQ ID NO: 3
  Synthetic Construct
  misc_feature 50,51: Joined by a phosphorothioate linkage
  misc_feature 1: Proprietary hydrazide modification at the 5′ end entitled the I-Linker

SEQ ID NO: 5 - DNA, 91 bp, Artificial Sequence
  Synthetic Construct
ccccaagaaccgaagtgctttgtcatagcttcctgatgacatatcataccaggccactgactgatgacagtaataggtgaaattttgccga

SEQ ID NO: 6 - DNA, 91 bp, Artificial Sequence
  Synthetic Construct
tcggcaaaatttcacctattactgtcatcagtcagtggcctggtatgatatgtcatcaggaagctatgacaaagcacttcggttcttgggg

SEQ ID NO: 7 - DNA, 100 bp, Artificial Sequence
  Synthetic Construct
atcatttgatcccaagaaccgaagtgctttgtcatagcttcctgatgacacatcataccaggccactgactgatgacagtaataggtgaaattttgccga

SEQ ID NO: 8 - DNA, 100 bp, Artificial Sequence
  Synthetic Construct
tcggcaaaatttcacctattactgtcatcagtcagtggcctggtatgatgtgtcatcaggaagctatgacaaagcacttcggttcttgggatcaaatgat